SEQUENCE LISTING (18 sequences)

SEQ ID NO: 1 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  MOD_RES (2)..(2): Any amino acid
  VARIANT (3)..(3): /replace=“Cys” or “Gln” or “Met” or “Ser” or “Thr” or “Gly” or “Ala” or “Ile” or “Leu” or “Pro” or “Val”
  MOD_RES (4)..(5): Any amino acid
  VARIANT (7)..(7): /replace=“Dap” or “N-AlkDap” or “N-HAlkDap”
  MOD_RES (12)..(13): Any amino acid
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CXNXXGCEDFYXXC

SEQ ID NO: 2 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CYNEFGCEDFYDIC

SEQ ID NO: 3-5 - skipped (3 entries)

SEQ ID NO: 6 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (2)..(2): /replace=“Met” or “Phe” or “Val”
  VARIANT (3)..(3): /replace=“Cys” or “Gln” or “Met” or “Ser” or “Thr” or “Gly” or “Ala” or “Ile” or “Leu” or “Pro” or “Val”
  VARIANT (4)..(4): /replace=“Cys” or “Gln” or “Met” or “Ser” or “Thr” or “Asp” or “Glu”
  VARIANT (5)..(5): /replace=“Trp” or “Tyr”
  VARIANT (7)..(7): /replace=“Dap” or “N-AlkDap” or “N-HAlkDap”
  VARIANT (12)..(12): /replace=“Glu”
  VARIANT (13)..(13): /replace=“Ala” or “Ile” or “Leu” or “Pro” or “Val”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CYNNFGCEDFYDGC

SEQ ID NO: 7 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (2)..(2): /replace=“Met” or “Phe” or “Val”
  VARIANT (3)..(3): /replace=“Gly”
  VARIANT (4)..(4): /replace=“Gln”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CYNEFGCEDFYDIC

SEQ ID NO: 8 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (2)..(2): /replace=“Met” or “Phe”
  VARIANT (3)..(3): /replace=“Gly”
  VARIANT (4)..(4): /replace=“Gln”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CYNEFGCEDFYDIC

SEQ ID NO: 9 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (2)..(2): /replace=“Met”
  VARIANT (4)..(4): /replace=“Gln”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CYNEFGCEDFYDIC

SEQ ID NO: 10 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CMNQFGCEDFYDIC

SEQ ID NO: 11 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CFGEFGCEDFYDIC

SEQ ID NO: 12 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CVNEFGCEDFYDIC

SEQ ID NO: 13 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CFNEFGCEDFYDIC

SEQ ID NO: 14 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CYNEYGCEDFYDIC

SEQ ID NO: 15 - protein, 14 aa, Artificial Sequence
  source: /note=“Description of Artificial Sequence: Synthetic peptide”
  VARIANT (1)..(1): /replace=“Dap” or “N-AlkDap” or “N-HAlkDap”
  VARIANT (7)..(7): /replace=“Dap” or “N-AlkDap” or “N-HAlkDap”
  VARIANT (14)..(14): /replace=“Dap” or “N-AlkDap” or “N-HAlkDap”
  MISC_FEATURE (1)..(14): /note=“Variant residues given in the sequence have no preference with respect to those in the annotations for variant positions”
  source: /note=“See specification as filed for detailed description of substitutions and preferred embodiments”
CYNEWGCEDFYDIC

SEQ ID NO: 16 - protein, 26 aa, Artificial Sequence
  source: /note=“Description of Artificial Sequence: Synthetic peptide”
  MOD_RES (1)..(1): beta-Ala
  MOD_RES (2)..(11): Sarcosine
  VARIANT (13)..(13): /replace=“Dap” or “N-AlkDap” or “N-HAlkDap”
  MOD_RES (14)..(14): D-amino acid
  MOD_RES (17)..(17): Fmoc-L-1-Naphthylalanine
  MOD_RES (18)..(18): D-amino acid
  VARIANT (19)..(19): /replace=“Dap” or “N-AlkDap” or “N-HAlkDap”
  MOD_RES (25)..(25): Fmoc-alpha-tert-butylglycine
  VARIANT (26)..(26): /replace=“Dap” or “N-AlkDap” or “N-HAlkDap”
  MISC_FEATURE (1)..(26): /note=“Variant residues given in the sequence have no preference with respect to those in the annotations for variant positions”
  source: /note=“See specification as filed for detailed description of substitutions and preferred embodiments”
AXXXXXXXXXXACANEAACEDFYDGC

SEQ ID NO: 17 - protein, 15 aa, Artificial Sequence
  source: /note=“Description of Artificial Sequence: Synthetic peptide”
  VARIANT (2)..(2): /replace=“Dap” or “N-AlkDap” or “N-HAlkDap”
  MOD_RES (3)..(3): D-amino acid
  MOD_RES (6)..(6): Fmoc-L-1-Naphthylalanine
  MOD_RES (7)..(7): D-amino acid
  VARIANT (8)..(8): /replace=“Dap” or “N-AlkDap” or “N-HAlkDap”
  MOD_RES (14)..(14): Fmoc-alpha-tert-butylglycine
  VARIANT (15)..(15): /replace=“Dap” or “N-AlkDap” or “N-HAlkDap”
  MISC_FEATURE (1)..(15): /note=“Variant residues given in the sequence have no preference with respect to those in the annotations for variant positions”
  source: /note=“See specification as filed for detailed description of substitutions and preferred embodiments”
ACANEAACEDFYDGC

SEQ ID NO: 18 - protein, 6 aa, Artificial Sequence
  source: /note=“Description of Artificial Sequence: Synthetic 6xHis tag”
HHHHHH

(19) United States
(12) Patent Application Publication
(10) Pub. No.: US 2019/0389907 A1
(43) Pub. Date: Dec. 26, 2019

(54) PEPTIDE LIGANDS FOR BINDING TO MT1-MMP

(71) Applicant: BicycleTx Limited, Cambridge (GB)

(72) Inventors: Daniel Teufel, Cambridge (GB); Gemma Mudd, Cambridge (GB); Silvia Pavan, Cambridge (GB)

(21) Appl. No.: 16/472,234

(22) PCT Filed: Dec. 20, 2017
(86) PCT No.: PCT/EP2017/083954

§ 371 Date: Jun. 21, 2019

(30) Foreign Application Priority Data
Dec. 23, 2016 (GB) 1622142.6
Aug. 23, 2017 (GB) 1713560.9

(51) Int. Cl. C07K 7/08; G01N 33/573; A61K 47/64

(52) U.S. Cl. CPC C07K 7/08; A61K 47/6415; A61K 47/64; G01N 33/573

(57) ABSTRACT

A peptide ligand specific for MT1-MMP comprising a polypeptide comprising two diaminopropionic acid (Dap) or N-alkyldiaminopropionic acid (N-AlkDap) residues, and a third residue selected from cysteine, Dap or N-AlkDap, separated by at least two loop sequences, and a molecular scaffold, the peptide being linked to the scaffold by covalent alkylamino linkages with the Dap or N-AlkDap residues of the polypeptide and by covalent thioether linkages with the cysteine when the third residue is cysteine, such that two polypeptide loops are formed on the molecular scaffold, wherein the peptide ligand comprises an amino acid sequence of formula (II): -A₁-X₁-U/O₂-X₃-X₄-G₅-A₂-E₆-D₇-F₈-Y₉-X₁₀-X₁₁-A₃- (SEQ ID NO: 1) (II) or a pharmaceutically acceptable salt thereof; wherein: A₁, A₂, and A₃ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of A₁, A₂, and A₃ is Dap, N-AlkDap or N-HAlkDap; X represents any amino acid residue; U represents a polar, uncharged amino acid residue selected from N, C, Q, M, S and T; and O represents a non-polar aliphatic amino acid residue selected from G, A, I, L, P and V.

### SEQUENCE LISTING

The instant application contains a Sequence Listing which has been submitted electronically in ASCII format and is hereby incorporated by reference in its entirety. Said ASCII copy, created on Mar. 21, 2018, is named P5685PC00_SL.txt and is 16 kilobytes in size.

### TECHNICAL FIELD

The present invention relates to peptide ligands showing high binding affinity to MT1-MMP. In particular, the invention relates to peptide ligands of this type having novel chemistries for forming two or more bonds between a peptide and a scaffold molecule.

### BACKGROUND OF THE INVENTION

Different research teams have previously tethered peptides to scaffold moieties by forming two or more thioether bonds between cysteine residues of the peptide and suitable functional groups of a scaffold molecule. For example, methods for the generation of candidate drug compounds by linking cysteine-containing peptides to a molecular scaffold as for example tris(bromomethyl) benzene are disclosed in WO 2004/077062 and WO 2006/078161.

The advantage of utilising cysteine thiols for generating covalent thioether linkages in order to achieve cyclisation resides is their selective and biorthogonal reactivity. Thiol-containing linear peptides may be cyclised with a thiol-reactive scaffold compound such as 1, 3, 5 tris-bromomethylbenzene (TBMB) to form Bicyclic Peptides, and the resultant product contains three thioethers at the benzylic locations. The overall reaction of the linear peptide with TBMB to form a looped bicyclic peptide with thioether linkages is shown in FIG. 1.

A need exists for alternative chemistries for coupling peptides to scaffold moieties to form looped peptide structures employing suitable replacements of the thioether moiety, thereby achieving compatibility with different peptides, changes in physiochemical properties such as improved solubility, changes in biodistribution and other advantages.

WO2011/018227 describes a method for altering the conformation of a first peptide ligand or group of peptide ligands, each peptide ligand comprising at least two reactive groups separated by a loop sequence covalently linked to a molecular scaffold which forms covalent bonds with said reactive groups, to produce a second peptide ligand or group of peptide ligands, comprising assembling said second derivative or group of derivatives from the peptide(s) and scaffold of said first derivative or group of derivatives, incorporating one of: (a) altering at least one reactive group; or (b) altering the nature of the molecular scaffold; or (c) altering the bond between at least one reactive group and the molecular scaffold; or any combination of (a), (b) or (c).

Our earlier published application WO2016/067035 and pending application GB1607827.1 filed on 4 May 2016 describe bicycle peptide ligands having high binding affinity for MT1-MMP. These applications further describe conjugates of the peptide ligands with therapeutic agents, in particular with cytotoxic agents. The entire disclosure of these applications is expressly incorporated herein.

### SUMMARY OF THE INVENTION

The present inventors have found that replacement of thioether linkages in looped peptides having affinity for MT1-MMP by alkylamino linkages results in looped peptide conjugates that display similar affinities to MT1-MMP as the corresponding conjugates made with all thioether linkages. The replacement of thioether linkages by alkylamino linkages is expected to result in improved solubility and/or improved oxidation stability of the conjugates according to the present invention.

Accordingly, in a first aspect the present invention provides a peptide ligand specific for MT1-MMP comprising a polypeptide comprising three residues selected from cysteine, L-2,3-diaminopropionic acid (Dap), N-beta-alkyl-L-2,3-diaminopropionic acid (N-AlkDap) and N-beta-haloalkyl-L-2,3-diaminopropionic acid (N-HAlkDap), the said three residues being separated by at least two loop sequences, and a molecular scaffold, the peptide being linked to the scaffold by covalent alkylamino linkages with the Dap or N-AlkDap or N-HAlkDap residues of the polypeptide and by thioether linkages with the cysteine residues of the polypeptide when the said three residues include cysteine, such that two polypeptide loops are formed on the molecular scaffold, wherein the peptide ligand comprises an amino acid sequence of formula (II):

(II)

(SEQ ID NO: 1)

-A₁-X₁-U/O₂-X₃-X₄-G₅-A₂-E₆-D₇-F₈-Y₉-X₁₀-X₁₁-A₃-

or a pharmaceutically acceptable salt thereof;
wherein:
A₁, A₂, and A₃ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of A₁, A₂, and A₃ is Dap, N-AlkDap or N-HAlkDap;
X represents any amino acid residue;
U represents a polar, uncharged amino acid residue selected from N, C, Q, M, S and T; and
O represents a non-polar aliphatic amino acid residue selected from G, A, I, L, P and V.

It can be seen that the derivatives of the invention comprise a peptide loop coupled to a scaffold by at least one alkylamino linkage to Dap or N-AlkDap of N-HAlkDap residues and up to two thioether linkages to cysteine. Suitably, A₁, A₂, and A₃ consist of one cysteine and two residues selected from Dap, N-AlkDap or N-HAlkDap. The prefix “alkyl” in N-AlkDap and N-HAlkDap refers to an alkyl group having from one to four carbon atoms, preferably methyl. The prefix “halo” is used in this context in its normal sense to signify alkyl groups having one or more, suitably one, fluoro-, chloro-, bromo- or iodo-substituents.

When cysteine is present, the thioether linkage(s) provides an anchor during formation of the cyclic peptides as explained further below. In these embodiments, the thioether linkage is suitably a central linkage of the bicyclic peptide conjugate, i.e. in the peptide sequence two residues forming alkylamino linkages in the peptide are spaced from and located on either side of a cysteine residue forming the thioether linkage. The looped peptide structure is therefore a Bicycle peptide conjugate having a central thioether linkage and two peripheral alkylamino linkages. In alternative embodiments, the thioether linkage is placed at the N-terminus or C-terminus of the peptides, the central linkage and the other terminal linkage being selected from Dap, N-AlkDap or N-HAlkDap.

In embodiments of the invention all three of A₁, A₂, and A₃ may suitably be Dap or N-AlkDap or N-HAlkDap. In these embodiments, the peptide ligands of the invention are suitably Bicycle conjugates having a central alkylamino linkage and two peripheral alkylamino linkages, the peptide forming two loops sharing the central alkylamino linkage. In these embodiments, A₁, A₂, and A₃ are suitably all selected from N-AlkDap or N-HAlkDap, most suitably N-AlkDap, because of favourable reaction kinetics with the alkylated Daps.

Suitably, X₁ is selected from any one of the following amino acids: Y, M, F or V, such as Y, M or F, in particular, Y or M, more particularly Y.

Suitably, U/O₂ is selected from a U, such as an N, or an O, such as a G.

Suitably, X₃ is selected from U or Z, wherein U represents a polar, uncharged amino acid residue selected from N, C, Q, M, S and T and Z represents a polar, negatively charged amino acid residue selected from D or E, in particular the U at position 3 is selected from Q or the Z at position 3 is selected from E.

Suitably, X₄ is selected from J, wherein J represents a non-polar aromatic amino acid residue selected from F, W and Y.

Suitably, X₁₀ is selected from Z, wherein Z represents a polar, negatively charged amino acid residue selected from D or E, such as D.

Suitably, X₁₁ is selected from O, wherein O represents a non-polar aliphatic amino acid residue selected from G, A, I, L, P and V, such as I.

Suitably, the Bicycle of formula (II) is a compound of formula (IIa):

(IIa)

(SEQ ID NO: 6

-A₁-Y/M/F/V-U/O-U/Z-J-G-A₂-E-D-F-Y-Z-O-A₃-

wherein U, O, J and Z are as defined hereinbefore; or
a compound of formula (IIb):

(IIb)

(SEQ ID NO: 7)

-A₁-Y/M/F/V-N/G-E/Q-F-G-A₂-E-D-F-Y-D-I-A₃-;

or
a compound of formula (IIc):

(IIc)

(SEQ ID NO: 8)

-A₁-Y/M/F-N/G-E/Q-F-G-A₂-E-D-F-Y-D-I-A₃-;

or
a compound of formula (IId):

(IId)

(SEQ ID NO: 9)

-A₁-Y/M-N-E/Q-F-G-A₂-E-D-F-Y-D-I-A₃-;

or
a compound of formula (IIe):

(IIe)

(SEQ ID NO: 2)

-A₁-Y-N-E-F-G-A₂-E-D-F-Y-D-I-A₃-(17-69-07).

Suitably, the Bicycle of formula (II) comprises a sequence selected from:

(SEQ ID NO: 2)

-A₁-Y-N-E-F-G-A₂-E-D-F-Y-D-I-A₃ (17-69-07);

(SEQ ID NO: 10)

-A₁-M-N-Q-F-G-A₂-E-D-F-Y-D-I-A₃ (17-69-12);

(SEQ ID NO: 11)

-A₁-F-G-E-F-G-A₂-E-D-F-Y-D-I-A₃ (17-69-02);

(SEQ ID NO: 12)

-A₁-V-N-E-F-G-A₂-E-D-F-Y-D-I-A₃ (17-69-03);

(SEQ ID NO: 13)

-A₁-F-N-E-F-G-A₂-E-D-F-Y-D-I-A₃ (17-69-04);

(SEQ ID NO: 14)

-A₁-Y-N-E-Y-G-A₂-E-D-F-Y-D-I-A₃;
and

(SEQ ID NO: 15)

-A₁-Y-N-E-W-G-A₂-E-D-F-Y-D-I-A₃,

such as:

(SEQ ID NO: 2)

-A₁-Y-N-E-F-G-A₂-E-D-F-Y-D-I-A₃ (17-69-07);
and

(SEQ ID NO: 10)

-A₁-M-N-Q-F-G-A₂-E-D-F-Y-D-I-A₃ (17-69-12),

in particular:

(SEQ ID NO: 2)

-A₁-Y-N-E-F-G-A₂-E-D-F-Y-D-I-A₃ (17-69-07),

most particularly:
the Dap homologues of 17-69-07-N241 designated as SEQ ID 16: ((bAla)-Sar10-AA₁(D-Ala)NE(1Nal)(D-Ala)A₂EDFYD(tBuGly)A₃;
and the Dap homologues of 17-69-07-N268 designated as SEQ ID 17: AA₁(D-Ala)NE(1Nal)(D-Ala)A₂EDFYD(tBuGly)A₃.

In all of the above sequences, A₁, A₂, and A₃ are as hereinbefore defined. Suitable and preferred types and positions of A₁, A₂, and A₃ are as hereinbefore defined.

In embodiments, the peptide ligand of the present invention additionally comprises one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more hydrophobic amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the α-carbon of one or more amino acid residues with another chemical group, and post-synthetic bioorthogonal modification of amino acids such as cysteine, lysine, glutamate and tyrosine with suitable amine, thiol, carboxylic acid and phenol-reactive reagents.

Suitably, these embodiments may comprise an N-terminal modification using suitable amino-reactive chemistry, and/or C-terminal modification using suitable carboxy-reactive chemistry. For example, the N-terminal modification may comprise the addition of a molecular spacer group which facilitates the conjugation of effector groups and retention of potency of the bicyclic peptide to its target. The spacer group is suitably an oligopeptide group containing from about 5 to about 30 amino acids, such as an Ala, G-Sar10-A group or bAla-Sar10-A group. Alternatively or additionally, the N-terminal and/or C-terminal modification comprises addition of a cytotoxic agent.

Further possible peptide modifications include a modification at amino acid position 1 and/or 9.

In embodiments, the peptide modification comprises replacement of one or more amino acid residues with one or more non-natural amino acid residues. For example, wherein the non-natural amino acid residue is substituted at position 4 and is selected from: 1-naphthylalanine; 2-naphthylalanine; 3,4-dichlorophenylalanine; and homophenylalanine, such as 1-naphthylalanine; 2-naphthylalanine; and 3,4-dichlorophenylalanine, in particular 1-naphthylalanine. Alternatively or additionally, the non-natural amino acid residue is substituted at position 9 and/or 11 and is selected from: 4-bromophenylalanine or pentafluoro-phenylalanine for position 9 and/or tert-butylglycine for position 11. In these embodiments, the non-natural amino acid residues, such as those present at position 9, may be selected from: 4-bromophenylalanine and/or the non-natural amino acid residues, such as those present at position 11, is selected from: tert-butylglycine.

In embodiments, the amino acid residue at position 1 is substituted for a D-amino acid, such as D-alanine. In other embodiments, the amino acid residue at position 5 is substituted for a D-amino acid, such as D-alanine or D-arginine

Suitably, the peptide ligand may comprise a plurality of the above mentioned modifications, such as 2, 3, 4 or 5 or more of the following modifications, such as all of the following 5 modifications: D-alanine at position 1 and/or 5, a 1-naphthylalanine at position 4, a 4-bromophenylalanine at position 9 and a tert-butylglycine at position 11.

In all of the peptide sequences defined herein, one or more tyrosine residues may be replaced by phenylalanine. This has been found to improve the yield of the bicycle peptide product during base-catalyzed coupling of the peptide to the scaffold molecule.

Suitably, the peptide ligand of the invention is a high affinity binder of the human, mouse and dog MT1-MMP hemopexin domain. Suitably the binding affinity kᵢ is less than about 100 nM, less than about 50 nM, less than about 25 nM, or less than about 10 nM.

Suitably, the peptide ligand of the invention is selective for MT1-MMP, but does not cross-react with MMP-1, MMP-2, MMP-15 and MMP-16. Suitably, the binding affinity ki with each of these ligands is greater than about 500 nM, greater than about 1000 nM, or greater than about 10000 nM.

Suitably, the scaffold comprises a (hetero)aromatic or (hetero)alicyclic moiety. Suitably, the scaffold comprises a tris-substituted (hetero)aromatic or (hetero)alicyclic moiety, for example a tris-methylene substituted (hetero)aromatic or (hetero)alicyclic moiety. The (hetero)aromatic or (hetero)alicyclic moiety is suitably a six-membered ring structure, preferably tris-substituted such that the scaffold has a 3-fold symmetry axis. Thus, in certain preferred embodiments, the scaffold is 1,3,5-tris-methylbenzene. In other preferred embodiments, the scaffold is a 1,3,5-tris-(acetamido)benzene group, which may be derived by coupling the peptide to 1,3,5-tris-(bromoacetamido)benzene (TBAB) as described further below.

In a second aspect, the present invention provides a peptide comprising an amino acid sequence of formula (II) as defined above in relation to the first aspect of the invention. Suitably, the peptide is suitable for making a peptide ligand according to the invention by linkage to a suitable scaffold molecule as described below. Suitably, the peptide is a linear peptide.

In a further aspect, the present invention provides a method of making a peptide ligand according to the first aspect of the invention, the method comprising: providing a peptide in accordance with the second aspect of the invention; providing a scaffold molecule having at least three reactive sites for forming alkylamino linkages with the side chain amino groups of the said cysteine and diaminopropionic acid or β-N-Alkyldiaminopropionic acid residues; and forming said alkylamino linkages between the peptide and the scaffold molecule.

The reactive sites are also suitable for forming thioether linkages with the —SH groups of cysteine in embodiments where the third residue is cysteine. The —SH group of cysteine is highly nucleophilic, and in these embodiments it is expected to react first with the electrophilic centres of the scaffold molecule to anchor the peptide to the scaffold molecule, whereafter the amino groups react with the remaining electrophilic centres of the scaffold molecule to form the looped peptide ligand.

In embodiments, the peptide has protecting groups on nucleophilic groups other than the amino groups and —SH groups (when present) intended for forming the alkylamino linkages

Suitably, the method of the invention comprises reacting, in a nucleophilic substitution reaction, the peptide as defined herein with a scaffold molecule having three or more leaving groups.

In alternative methods, the compounds of the present invention could be made converting two or more side chain groups of the peptide to leaving groups, followed by reacting the peptide, in a nucleophilic substitution reaction, with a scaffold molecule having two or more amino groups.

The nucleophilic substitution reactions may be performed in the presence of a base, for example where the leaving group is a conventional anionic leaving group. The present inventors have found that the yields of cyclised peptide ligands can be greatly increased by suitable choice of solvent and base for the nucleophilic substitution reaction, and furthermore that the preferred solvent and base are different from the prior art solvent and base combinations that involve only the formation of thioether linkages. In particular, the present inventors have found that improved yields are achieved when using a trialkylamine base, i.e. a base of formula NR₁R₂R₃, wherein R₁, R₂ and R₃ are independently C1-C5 alkyl groups, suitably C2-C4 alkyl groups, in particular C2-C3 alkyl groups. Especially suitable bases are triethylamine and diisopropylethylamine (DIPEA). These bases have the property of being only weakly nucleophilic, and it is thought that this property accounts for the fewer side reactions and higher yields observed with these bases. The present inventors have further found that the preferred solvents for the nucleophilic substitution reaction are polar and protic solvents, in particular MeCN/H₂O (50:50).

In a further aspect, the present invention provides a drug conjugate comprising the peptide ligand according to the invention conjugated to one or more effector and/or functional groups such as a cytotoxic agent or a metal chelator.

Suitably, the conjugate has the cytotoxic agent linked to the peptide ligand by a cleavable bond, such as a disulphide bond. Suitably, the cytotoxic agent is selected from DM1 or MMAE.

In embodiments, the drug conjugate has the following structure:

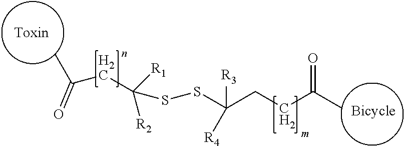

wherein: R₁, R₂, R₃ and R₄ represent hydrogen or C1-C6 alkyl groups;
Toxin refers to any suitable cytotoxic agent;
Bicycle represents the looped peptide structure;
n represents an integer selected from 1 to 10; and
m represents an integer selected from 0 to 10.

Suitably, either: R₁, R₂, R₃ and R₄ are all H; or R₁, R₂, R₃ are all H and R₄=methyl; or R₁, R₂=methyl and R₃, R₄=H; or R₁, R₃=methyl and R₂, R₄=H; or R₁, R₂=H and R₃, R₄=C1-C6 alkyl.

The linker between the toxin and the bicycle peptide may comprise a triazole group formed by click-reaction between an azide-functionalized toxin and an alkyne-functionalized bicycle peptide structure (or vice-versa). In other embodiments, the bicycle peptide may contain an amide linkage formed by reaction between a carboxylate-functionalized toxin and the N-terminal amino group of the bicycle peptide.

The linker between the toxin and the bicycle peptide may comprise a cathepsin-cleavable group to provide selective release of the toxin within the target cells. A suitable cathepsin-cleavable group is valine-citrulline.

The linker between the toxin and the bicycle peptide may comprise one or more spacer groups to provide the desired functionality, e.g. binding affinity or cathepsin cleavability, to the conjugate. A suitable spacer group is para-amino benzyl carbamate (PABC) which may be located intermediate the valine-citrulline group and the toxin moiety.

Thus, in embodiments, the bicycle peptide-drug conjugate may have the following structure made up of Toxin-PABC-cit-val-triazole-Bicycle:

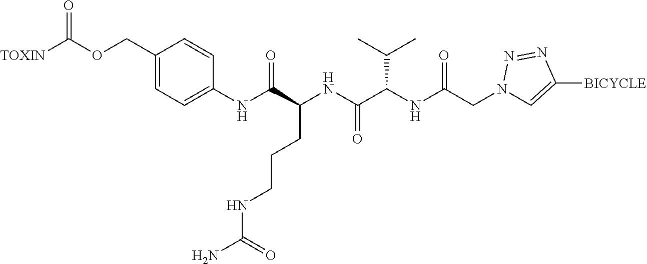

In further embodiments, the bicycle peptide-drug conjugate may have the following structure made up of Toxin-PABC-cit-val-dicarboxylate-Bicycle:

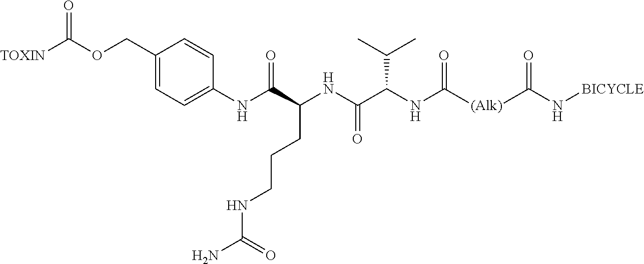

Wherein (alk) is an alkylene group of formula CₙH₂ₙ wherein n is from 1 to 10 and may be linear or branched, suitably (alk) is n-propylene or n-butylene.

In another aspect, the invention further provides a kit comprising at least a peptide ligand or conjugate according to the present invention.

In a still further aspect, the present invention provides a composition comprising a peptide ligand or conjugate of the present invention, and a pharmaceutically acceptable carrier, diluent or excipient.

Moreover, the present invention provides a method for the treatment of disease using a peptide ligand, conjugate, or a composition according to the present invention. Suitably, the disease is a neoplastic disease, such as cancer.

In a further aspect, the present invention provides a method for the diagnosis, including diagnosis of disease using a peptide ligand, or a composition according to the present invention. Thus in general the binding of an analyte to a peptide ligand may be exploited to displace an agent, which leads to the generation of a signal on displacement. For example, binding of analyte (second target) can displace an enzyme (first target) bound to the peptide ligand providing the basis for a binding assay, especially if the enzyme is held to the peptide ligand through its active site.

### BRIEF DESCRIPTION OF THE DRAWINGS

FIG. 1 shows a reaction scheme for preparation of thioether-linked bicyclic peptide ligands according to the prior art;

FIG. 2 shows a thioether-linked bicyclic peptide ligand according to the prior art designated as 17-69-07-N241

FIG. 3 shows a first secondary amino-linked bicyclic peptide ligand according to the present invention;

FIG. 4 shows a tertiary N-methyl amino-linked bicyclic peptide ligand according to the present invention;

FIG. 5 shows a third secondary amino-linked bicyclic peptide ligand according to the present invention, which is the Dap analogue of 17-69-07-N241;

FIG. 6 shows a fourth secondary amino-linked bicyclic peptide ligand according to the present invention, cyclised with the TBAB scaffold;

FIG. 7 shows competitive affinity binding assay data against MT1-MMP for the derivative of FIG. 3;

FIG. 8 shows competitive affinity binding assay data against MT1-MMP for the derivative of FIG. 4; and

FIG. 9 shows competitive affinity binding assay data against MT1-MMP for a further derivative according to the invention.

FIG. 10 shows the schematic structures of certain bicycle peptide-TBMB derivatives in accordance with the present invention;

FIG. 11 shows the schematic structures of further bicycle peptide-TBMB derivatives in accordance with the present invention;

FIG. 12 shows the schematic structures of further bicycle peptide-TBMB derivatives in accordance with the present invention;

FIG. 13 shows the schematic structures of further bicycle peptide-TBMB derivatives in accordance with the present invention;

FIG. 14 shows a reaction scheme for preparation of a bicycle peptide-drug conjugate according to the invention by a click reaction to form a triazole linkage;

FIG. 15 shows a reaction scheme for preparation of a bicycle peptide-drug conjugate according to the invention by having an amido linkage;

FIG. 16 shows tumor volume and body mass over time for Balb/c nude mice having HT1020 tumor cell tumors after treatment with a bicycle peptide-drug conjugate according to the invention;

FIG. 17 shows tumor volume and body mass over time for Balb/c nude mice having HT1020 tumor cell tumors after treatment with a further bicycle peptide-drug conjugate according to the invention;

FIG. 18 shows tumor volume and body mass over time for Balb/c nude mice having HT1020 tumor cell tumors after treatment with a further bicycle peptide-drug conjugate according to the invention;

FIG. 19 shows tumor volume and body mass over time for Balb/c nude mice having HT1020 tumor cell tumors after treatment with a further bicycle peptide-drug conjugate according to the invention;

FIG. 20 shows tumor volume and body mass over time for Balb/c nude mice having HT1020 tumor cell tumors after treatment with a further bicycle peptide-drug conjugate according to the invention.

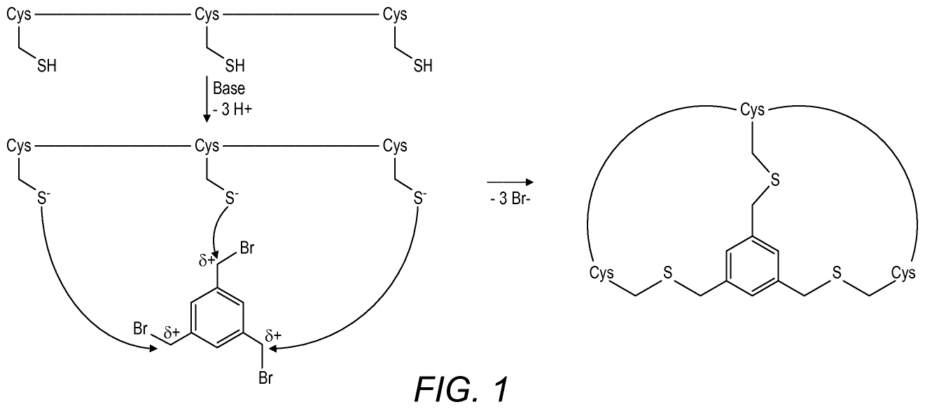

### DETAILED DESCRIPTION OF THE INVENTION

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

The present invention provides a looped peptide structure as defined in claim 1 comprising two peptide loops subtended between three linkages on the molecular scaffold, the central linkage being common to the two loops. The central linkage suitably is a thioether linkage formed to a cysteine residue of the peptide, or it is an alkylamino linkage formed to a Dap or N-AlkDap residue of the peptide. The two outer linkages are suitably alkylamino linkages formed to Dap or N-AlkDap residues of the peptide, or one of the outer linkages may be a thioether linkage formed to a cysteine residue of the peptide.

It will be appreciated by the skilled person that the X at positions 1, 3, 4, 10 and 11 of formula (II) may represent any amino acid following the results of an alanine scan and selection outputs which permits well tolerated substitutions at these positions.

In one embodiment, the X at position 1 of formula (II) is selected from any one of the following amino acids: Y, M, F or V. In a further embodiment, the X at position 1 of formula (II) is selected from Y, M or F. In a yet further embodiment, the X at position 1 of formula (II) is selected from Y or M. In a still yet further embodiment, the X at position 1 of formula (II) is selected from Y.

In one embodiment, the U/O at position 2 of formula (II) is selected from a U, such as an N. In an alternative embodiment, the U/O at position 2 of formula (II) is selected from an O, such as a G.

In one embodiment, the X at position 3 of formula (II) is selected from U or Z, wherein U represents a polar, uncharged amino acid residue selected from N, C, Q, M, S and T and Z represents a polar, negatively charged amino acid residue selected from D or E. In a further embodiment, the U at position 3 of formula (II) is selected from Q. In an alternative embodiment, the Z at position 3 of formula (II) is selected from E.

In one embodiment, the X at position 4 of formula (II) is selected from J, wherein J represents a non-polar aromatic amino acid residue selected from F, W and Y. In a further embodiment, the J at position 4 of formula (II) is selected from F. In alternative embodiment, the J at position 4 of formula (II) is selected from Y. In alternative embodiment, the J at position 4 of formula (II) is selected from W.

In one embodiment, the X at position 10 of formula (II) is selected from Z, wherein Z represents a polar, negatively charged amino acid residue selected from D or E. In one embodiment, the Z at position 10 of formula (II) is selected from D.

In one embodiment, the X at position 11 of formula (II) is selected from O, wherein 0 represents a non-polar aliphatic amino acid residue selected from G, A, I, L, P and V. In one embodiment, the O at position 11 of formula (II) is selected from I.

In one embodiment, the compound of formula (II) is a compound of formula (IIa):

(IIa)

(SEQ ID NO: 6)

-A₁-Y/M/F/V-U/O-U/Z-J-G-A₂-E-D-F-Y-Z-O-A₃-;

wherein U, O, J and Z are as defined hereinbefore.

In one embodiment, the compound of formula (II) is a compound of formula (IIb):

(IIb)

(SEQ ID NO: 7)

-A₁-Y/M/F/V-N/G-E/Q-F-G-A₂-E-D-F-Y-D-I-A₃-.

In one embodiment, the compound of formula (II) is a compound of formula (IIc):

(IIc)

(SEQ ID NO: 8)

-A₁-Y/M/F-N/G-E/Q-F-G-A₂-E-D-F-Y-D-I-A₃-.

In one embodiment, the compound of formula (II) is a compound of formula (IId):

(IId)

(SEQ ID NO: 9)

-A₁-Y/M-N-E/Q-F-G-A₂-E-D-F-Y-D-I-A₃-.

In one embodiment, the compound of formula (II) is a compound of formula (IIe):

(IIe)

(SEQ ID NO: 2)

-A₁-Y-N-E-F-G-A₂-E-D-F-Y-D-I-A₃-(17-69-07).

In a yet further embodiment, the peptide of formula (II) comprises a sequence selected from:

(SEQ ID NO: 2)

-A₁-Y-N-E-F-G-A₂-E-D-F-Y-D-I-A₃-(17-69-07);

(SEQ ID NO: 10)

-A₁-M-N-Q-F-G-A₂-E-D-F-Y-D-I-A₃-(17-69-12);

(SEQ ID NO: 11)

-A₁-F-G-E-F-G-A₂-E-D-F-Y-D-I-A₃-(17-69-02);

(SEQ ID NO: 12)

-A₁-V-N-E-F-G-A₂-E-D-F-Y-D-I-A₃-(17-69-03);

(SEQ ID NO: 13)

-A₁-F-N-E-F-G-A₂-E-D-F-Y-D-I-A₃-(17-69-04);

(SEQ ID NO: 14)

-A₁-Y-N-E-Y-G-A₂-E-D-F-Y-D-I-A₃-(17-69-07-N057);
and

(SEQ ID NO: 15)

-A₁-Y-N-E-W-G-A₂-E-D-F-Y-D-I-A₃-(17-69-44-N002).

The peptides of this embodiment were identified to be potent candidates following affinity maturation against the hemopexin domain of MT1-MMP.

In a still yet further embodiment, the peptide of formula (II) comprises a sequence selected from:

(SEQ ID NO: 2)

-A₁-Y-N-E-F-G-A₂-E-D-F-Y-D-I-A₃-(17-69-07);
and

(SEQ ID NO: 10)

-A₁-M-N-Q-F-G-A₂-E-D-F-Y-D-I-A₃-(17-69-12).

The peptides of this embodiment were identified to be the highest affinity candidates following affinity maturation against the hemopexin domain of MT1-MMP, synthesis of the core bicycle sequences, and quantitative measurement of affinities using competition experiments.

In a still yet further embodiment, the peptide of formula (II) comprises a sequence selected from -A₁-Y-N-E-F-G-A₂-E-D-F-Y-D-I-A₃- (17-69-07) (SEQ ID NO: 2). The peptide of this embodiment was identified to be the most potent, and stable member of the family of peptide ligands within formula (II).

In a still yet further embodiment, the peptide of formula (II) comprises a sequence selected from:
(bAla)-Sar10-AA₁(D-Ala)NE(1Nal)(D-Ala)A₂EDFYD(tBuGly)A₃,); or
AA₁ (D-Ala)NE(1Nal)(D-Ala)A₂EDFYD(tBuGly)A₃),
   respectively, where the N-terminus is suitably present as the free amino group, and the C-terminus is suitably amidated.

In all of the above sequences, A₁, A₂, and A₃ are as hereinbefore defined. Suitable and preferred types and positions of A₁, A₂, and A₃ are as hereinbefore defined.

In one embodiment, certain peptides of formula (II) are fully cross-reactive with murine, dog, cynomolgus and human MT1-MMP. In a further embodiment, the specifically exemplified peptide ligands of the invention are fully cross-reactive with murine, dog, cynomolgus and human MT1-MMP. For example, both non-stabilised and stabilised derivatives of 17-69-07 (i.e. 17-69-07-N219, 17-69-07-N241 and 17-69-07-N268) are fully cross reactive.

In a yet further embodiment, the peptide of formula (II) is selective for MT1-MMP, but does not cross-react with MMP-1, MMP-2, MMP-15 and MMP-16. The 17-69-07 core sequence, and the stabilised variant 17-69-07-N258, are uniquely selective for MT1-MMP. Suitably the binding affinity kᵢ for MT1-MMP is less than about 100 nM, less than about 50 nM, less than about 25 nM, or less than about 10 nM. Suitably, the binding affinity ki with MMP-1, MMP-2, MMP-15 and MMP-16 is greater than about 500 nM, greater than about 1000 nM, or greater than about 10000 nM.

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyn-group bearing amino acids that allow functionalisation with alkyn or azide-bearing moieties, respectively.

In one embodiment, the modified derivative comprises a modification at amino acid position 1 and/or 9. These positions, especially where tyrosine is present, are most susceptible to proteolytic degradation.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as C) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target. The spacer group is suitably an oligopeptide group containing from about 5 to about 30 amino acids, such as an Ala, G-Sar10-A or bAla-Sar10-A group. In one embodiment, the spacer group is selected from bAla-Sar10-A (i.e. 17-69-07-N241). Addition of these spacer groups to the bicyclic peptide 17-69-07 does not alter potency to the target protein.

In a further embodiment, the modified derivative comprises a C-terminal modification. In a further embodiment, the C-terminal modification comprises an amide group. In this embodiment, the C-terminal cysteine group (the group referred to herein as C) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, C□-disubstituted derivatives (for example, aminoisobutyric acid, Aib), and cyclo amino acids, a simple derivative being amino-cyclopropylcarboxylic acid.

In one embodiment, the non-natural amino acid residue is substituted at position 4. A number of non-natural amino acid residues are well tolerated at this position. In a further embodiment, the non-natural amino acid residues, such as those present at position 4, are selected from: 1-naphthylalanine; 2-naphthylalanine; cyclohexylglycine, phenylglycine; tert-butylglycine; 3,4-dichlorophenylalanine; cyclohexylalanine; and homophenylalanine

In a yet further embodiment, the non-natural amino acid residues, such as those present at position 4, are selected from: 1-naphthylalanine; 2-naphthylalanine; and 3,4-dichlorophenylalanine. These substitutions enhance the affinity compared to the unmodified wildtype sequence.

In a yet further embodiment, the non-natural amino acid residues, such as those present at position 4, are selected from: 1-naphthylalanine. This substitution provided the greatest level of enhancement of affinity (greater than 7 fold) compared to wildtype.

In one embodiment, the non-natural amino acid residue is introduced at position 9 and/or 11. A number of non-natural amino acid residues are well tolerated at these positions.

In a further embodiment, the non-natural amino acid residues, such as those present at position 9, are selected from: 4-bromophenylalanine, pentafluoro-phenylalanine, such as 4-bromophenylalanine.

In a yet further embodiment, the non-natural amino acid residues, such as those present at position 11, is selected from: tert-butylglycine. Enhancement of activity and strong protection of the vicinal amino acid backbone from proteolytic hydrolysis is achieved by steric obstruction.

In one embodiment, the modified derivative comprises a plurality of the above mentioned modifications, such as 2, 3, 4 or 5 or more modifications. In a further embodiment, the modified derivative comprises 2, 3, 4 or 5 or more of the following modifications, such as all of the following 5 modifications: D-alanine at position 1 and 5, a 1-naphthylalanine at position 4, a 4-bromophenylalanine at position 9 and a tert-butylglycine at position 11. This multi-substitution is tolerated in concert with potency which is superior to wildtype. In a yet further embodiment, the modified derivative comprises the following modifications: D-alanine at position 1 and 5, a 1-naphthylalanine at position 4 and a tert-butylglycine at position 11. This multi-substitution is tolerated in concert with potency which is superior to wildtype.

In one embodiment, the modified derivative comprises the addition of a spacer group.

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise □-turn conformations (Tugyi et al (2005) PNAS, 102(2), 413-418).

In all of the peptide sequences defined herein, one or more tyrosine residues may be replaced by phenylalanine. This has been found to improve the yield of the bicycle peptide product during base-catalyzed coupling of the peptide to the scaffold molecule.

In a further embodiment, the amino acid residue at position 1 is substituted for a D-amino acid, such as D-alanine. This substitution achieves retention of potency without the consequent degradation.

In a further embodiment, the amino acid residue at position 5 is substituted for a D-amino acid, such as D-alanine or D-arginine. This substitution achieves retention of potency without the consequent degradation.

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.
   (for reviews see Gentilucci et al, Curr. Pharmaceutical Design, (2010), 16, 3185-203, and Nestor et al, Curr. Medicinal Chem (2009), 16, 4399-418).

The present invention includes all pharmaceutically acceptable (radio)isotope-labeled compounds of the invention, i.e. compounds of formula (II), wherein one or more atoms are replaced by atoms having the same atomic number, but an atomic mass or mass number different from the atomic mass or mass number usually found in nature, and compounds of formula (II), wherein metal chelating groups are attached (termed “effector”) that are capable of holding relevant (radio)isotopes, and compounds of formula (I), wherein certain functional groups are covalently replaced with relevant (radio)isotopes or isotopically labelled functional groups.

Examples of isotopes suitable for inclusion in the compounds of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled compounds of formula (II), for example, those incorporating a radioactive isotope, are useful in drug and/or substrate tissue distribution studies, and to clinically assess the presence and/or absence of the MT1-MMP target on diseased tissues such as tumours and elsewhere. The compounds of formula (II) can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, a N, can be useful in Positron Emission Topography (PET) studies for examining target occupancy.

Incorporation of isotopes into metal chelating effector groups, such as ⁶⁴Cu, ⁶⁷Ga, ⁶⁸Ga, and ¹⁷⁷Lu can be useful for visualizing tumour specific antigens employing PET or SPECT imaging

Incorporation of isotopes into metal chelating effector groups, such as, but not limited to ⁹⁰Y, ¹⁷⁷Lu, and ²¹³Bi, can present the option of targeted radiotherapy, whereby metal-chelator-bearing compounds of formula (II) carry the therapeutic radionuclide towards the target protein and site of action.

Isotopically-labeled compounds of formula (II) can generally be prepared by conventional techniques known to those skilled in the art or by processes analogous to those described in the accompanying Examples using an appropriate isotopically-labeled reagent in place of the non-labeled reagent previously employed.

Specificity, in the context herein, refers to the ability of a ligand to bind or otherwise interact with its cognate target to the exclusion of entities which are similar to the target. For example, specificity can refer to the ability of a ligand to inhibit the interaction of a human enzyme, but not a homologous enzyme from a different species. Using the approach described herein, specificity can be modulated, that is increased or decreased, so as to make the ligands more or less able to interact with homologues or paralogues of the intended target. Specificity is not intended to be synonymous with activity, affinity or avidity, and the potency of the action of a ligand on its target (such as, for example, binding affinity or level of inhibition) are not necessarily related to its specificity.

Binding activity, as used herein, refers to quantitative binding measurements taken from binding assays, for example as described herein. Therefore, binding activity refers to the amount of peptide ligand which is bound at a given target concentration.

Multispecificity is the ability to bind to two or more targets. Typically, binding peptides are capable of binding to a single target, such as an epitope in the case of an antibody, due to their conformational properties. However, peptides can be developed which can bind to two or more targets; dual specific antibodies, for example, as known in the art as referred to above. In the present invention, the peptide ligands can be capable of binding to two or more targets and are therefore multispecific. Suitably, they bind to two targets, and are dual specific. The binding may be independent, which would mean that the binding sites for the targets on the peptide are not structurally hindered by the binding of one or other of the targets. In this case, both targets can be bound independently. More generally, it is expected that the binding of one target will at least partially impede the binding of the other.

There is a fundamental difference between a dual specific ligand and a ligand with specificity which encompasses two related targets. In the first case, the ligand is specific for both targets individually, and interacts with each in a specific manner. For example, a first loop in the ligand may bind to a first target, and a second loop to a second target. In the second case, the ligand is non-specific because it does not differentiate between the two targets, for example by interacting with an epitope of the targets which is common to both.

In the context of the present invention, it is possible that a ligand which has activity in respect of, for example, a target and an orthologue, could be a bispecific ligand. However, in one embodiment the ligand is not bispecific, but has a less precise specificity such that it binds both the target and one or more orthologues. In general, a ligand which has not been selected against both a target and its orthologue is less likely to be bispecific due to the absence of selective pressure towards bispecificity. The loop length in the bicyclic peptide may be decisive in providing a tailored binding surface such that good target and orthologue cross-reactivity can be obtained, while maintaining high selectivity towards less related homologues.

If the ligands are truly bispecific, in one embodiment at least one of the target specificities of the ligands will be common amongst the ligands selected, and the level of that specificity can be modulated by the methods disclosed herein. Second or further specificities need not be shared, and need not be the subject of the procedures set forth herein.

The molecular scaffold is any molecule which is able to connect the peptide at multiple points to impart one or more structural features to the peptide. Preferably, the molecular scaffold comprises at least three attachment points for the peptide, referred to as scaffold reactive groups. These groups are capable of reacting with the Dap or N-AlkDap or cysteine (when present) residues (on the peptide to form stable, covalent alkylamino and thioether linkages. Preferred structures for molecular scaffolds are described below.

The compounds of the invention thus comprise, consist essentially of, or consist of, the peptide covalently bound to a molecular scaffold. The term “scaffold” or “molecular scaffold” herein refers to a chemical moiety that is bonded to the peptide at the alkylamino linkages and thioether linkage (when the third residue is cysteine) in the compounds of the invention. The term “scaffold molecule” or “molecular scaffold molecule” herein refers to a molecule that is capable of being reacted with a peptide or peptide ligand to form the derivatives of the invention having alkylamino and, in certain embodiments, also thioether bonds. Thus, the scaffold molecule has the same structure as the scaffold moiety except that respective reactive groups (such as leaving groups) of the molecule are replaced by alkylamino and thioether bonds to the peptide in the scaffold moiety.

The molecular scaffold molecule is any molecule which is able to connect the peptide at multiple points to form the thioether and alkylamino bonds to the peptide. It is not a cross-linker, in that it does not normally link two peptides; instead, it provides two or more attachment points for a single peptide. The molecular scaffold molecule comprises at least three attachment points for the peptide, referred to as scaffold reactive groups. These groups are capable of reacting with —SH and amino groups on the peptide to form the thioether and alkylamino linkages. Thus, the molecular scaffold represents the scaffold moiety up to but not including the thioether and alkylamino linkages in the conjugates of the invention. The scaffold molecule has the structure of the scaffold, but with reactive groups at the locations of the thioether and alkylamino bonds in the conjugate of the invention.

Suitably, the scaffold comprises, consists essentially of, or consists of a (hetero)aromatic or (hetero)alicyclic moiety.

As used herein, “(hetero)aryl” is meant to include aromatic rings, for example, aromatic rings having from 4 to 12 members, such as phenyl rings. These aromatic rings can optionally contain one or more heteroatoms (e.g., one or more of N, O, S, and P), such as thienyl rings, pyridyl rings, and furanyl rings. The aromatic rings can be optionally substituted. “(hetero)aryl” is also meant to include aromatic rings to which are fused one or more other aryl rings or non-aryl rings. For example, naphthyl groups, indole groups, thienothienyl groups, dithienothienyl, and 5,6,7,8-tetrahydro-2-naphthyl groups (each of which can be optionally substituted) are aryl groups for the purposes of the present application. As indicated above, the aryl rings can be optionally substituted. Suitable substituents include alkyl groups (which can optionally be substituted), other aryl groups (which may themselves be substituted), heterocyclic rings (saturated or unsaturated), alkoxy groups (which is meant to include aryloxy groups (e.g., phenoxy groups)), hydroxy groups, aldehyde groups, nitro groups, amine groups (e.g., unsubstituted, or mono- or di-substituted with aryl or alkyl groups), carboxylic acid groups, carboxylic acid derivatives (e.g., carboxylic acid esters, amides, etc.), halogen atoms (e.g., Cl, Br, and I), and the like.

As used herein, “(hetero)alicyclic” refers to a homocyclic or heterocyclic saturated ring. The ring can be unsubstituted, or it can be substituted with one or more substituents. The substituents can be saturated or unsaturated, aromatic or nonaromatic, and examples of suitable substituents include those recited above in the discussion relating to substituents on alkyl and aryl groups. Furthermore, two or more ring substituents can combine to form another ring, so that “ring”, as used herein, is meant to include fused ring systems.

Suitably, the scaffold comprises a tris-substituted (hetero)aromatic or (hetero)alicyclic moiety, for example a tris-methylene substituted (hetero)aromatic or (hetero)alicyclic moiety. The (hetero)aromatic or (hetero)alicyclic moiety is suitably a six-membered ring structure, preferably tris-substituted such that the scaffold has a 3-fold symmetry axis.

In embodiments, the scaffold is a tris-methylene (hetero)aryl moiety, for example a 1,3,5-tris methylene benzene moiety. In these embodiments, the corresponding scaffold molecule suitably has a leaving group on the methylene carbons. The methylene group then forms the Ri moiety of the alkylamino linkage as defined herein. In these methylene-substituted (hetero)aromatic compounds, the electrons of the aromatic ring can stabilize the transition state during nucleophilic substitution. Thus, for example, benzyl halides are 100-1000 times more reactive towards nucleophilic substitution than alkyl halides that are not connected to a (hetero)aromatic group.

In these embodiments, the scaffold and scaffold molecule have the general formula:

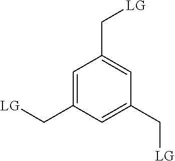

Where LG represents a leaving group as described further below for the scaffold molecule, or LG (including the adjacent methylene group forming the Ri moiety of the alkylamino group) represents the alkylamino linkage to the peptide in the conjugates of the invention.

In embodiments, the group LG above may be a halogen such as, but not limited to, a bromine atom, in which case the scaffold molecule is 1,3,5-Tris(bromomethyl)benzene (TBMB). Another suitable molecular scaffold molecule is 2,4,6-tris(bromomethyl) mesitylene. It is similar to 1,3,5-tris(bromomethyl) benzene but contains additionally three methyl groups attached to the benzene ring. In the case of this scaffold, the additional methyl groups may form further contacts with the peptide and hence add additional structural constraint. Thus, a different diversity range is achieved than with 1,3,5-Tris(bromomethyl)benzene.

Another preferred molecule for forming the scaffold for reaction with the peptide by nucleophilic substitution is 1,3,5-tris(bromoacetamido)benzene (TBAB):

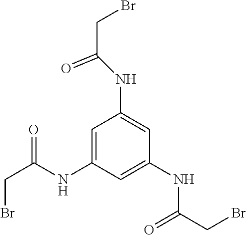 [SMILES: O=C(CBr)Nc1cc(NC(=O)CBr)cc(NC(=O)CBr)c1]

In other embodiments the molecular scaffold may have a tetrahedral geometry such that reaction of four functional groups of the encoded peptide with the molecular scaffold generates not more than two product isomers. Other geometries are also possible; indeed, an almost infinite number of scaffold geometries is possible, leading to greater possibilities for peptide ligand diversification.

The peptides used to form the ligands of the invention comprise Dap or N-AlkDap or N-HAlkDap residues for forming alkylamino linkages to the scaffold. The structure of diaminopropionic acid is analogous to and isosteric that of cysteine that has been used to form thioether bonds to the scaffold in the prior art, with replacement of the terminal —SH group of cysteine by —NH₂:

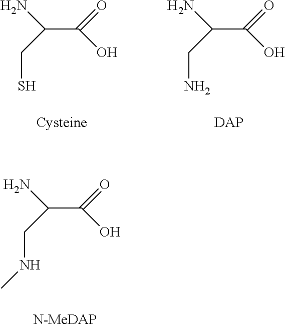

The term “alkylamino” is used herein in its normal chemical sense to denote a linkage consisting of NH or N(R₃) bonded to two carbon atoms, wherein the carbon atoms are independently selected from alkyl, alkylene, or aryl carbon atoms and R₃ is an alkyl group. Suitably, the alkylamino linkages of the invention comprise an NH moiety bonded to two saturated carbon atoms, most suitably methylene (—CH₂—) carbon atoms. The alkylamino linkages of the invention have general formula:

S—R₁—N(R₃)—R₂—P

### Wherein:

S represents the scaffold core, e.g. a (hetero)aromatic or (hetero)alicyclic ring as explained further below;
R₁ is C1 to C3 alkylene groups, suitably methylene or ethylene groups, and most suitably methylene (CH₂);
R₂ is the methylene group of the Dap or N-AlkDap side chain
R₃ is C1-4 alkyl including branched alkyl and cycloalkyl, for example methyl, or H; and
P represents the peptide backbone, i.e. the R₂ moiety of the above linkage is linked to the carbon atom in the peptide backbone adjacent to a carboxylic carbon of the Dap or N-AlkDap residue.

Certain bicyclic peptides of formula (II) have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes; and
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent.

It will be appreciated that salt forms are within the scope of this invention, and references to bicyclic peptide compounds of formula (II) include the salt forms of said compounds.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydroiodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of formula (II) contain an amine function, these may form quaternary ammonium salts, for example by reaction with an alkylating agent according to methods well known to the skilled person. Such quaternary ammonium compounds are within the scope of formula (II).

Several conjugated peptides may be incorporated together into the same molecule according to the present invention. For example two such peptide conjugates of the same specificity can be linked together via the molecular scaffold, increasing the avidity of the derivative for its targets. Alternatively, in another embodiment a plurality of peptide conjugates are combined to form a multimer. For example, two different peptide conjugates are combined to create a multispecific molecule. Alternatively, three or more peptide conjugates, which may be the same or different, can be combined to form multispecific derivatives. In one embodiment multivalent complexes may be constructed by linking together the molecular scaffolds, which may be the same or different.

In a further aspect, the present invention provides a method of making a peptide ligand according to the present invention, the method comprising: providing a peptide according to the invention and a scaffold molecule; and forming the thioether (when the third residue is cysteine) and alkylamino linkages between the peptide and the scaffold molecule.

The details of the scaffold molecule and the peptide are suitably as described above in relation to the first aspect of the invention.

The peptides for use in the methods of the invention can be made using conventional solid-phase synthesis from amino acid starting materials, which may include appropriate protecting groups as described herein. These methods for making peptides are well known in the art.

Suitably, the peptide has protecting groups on nucleophilic groups other than the —SH and amine groups intended for forming the alkylamino linkages. The nucleophilicity of amino acid side chains has been subject to several studies, and listed in descending order: thiolate in cysteines, amines in Lysine, secondary amine in Histidine and Tryptophan, guanidino amines in Arginine, hydroxyls in Serine/Threonine, and finally carboxylates in aspartate and glutamate. Accordingly, in some cases it may be necessary to apply protecting groups to the more nucleophilic groups on the peptide to prevent undesired side reactions with these groups.

In embodiments, the method of the invention comprises: synthesising a peptide having protecting groups on nucleophilic groups other than the amine groups intended for forming the alkylamino linkages and second protecting groups on the amine groups intended for forming alkylamino linkages, wherein the protecting groups on the amine groups intended for forming alkylamino linkages can be removed under conditions different than for the protecting groups on the other nucleophilic groups, followed by treating the peptide under conditions selected to deprotect the amine groups intended for forming alkylamino linkages without deprotecting the other nucleophilic groups. The coupling reaction to the scaffold is then performed, followed by removal of the remaining protecting groups to yield the peptide conjugate.

Suitably, the method of the invention comprises reacting, in a nucleophilic substitution reaction, the peptide having the reactive side chain —SH and amine groups, with a scaffold molecule having three or more leaving groups.

The term “leaving group” herein is used in its normal chemical sense to mean a moiety capable of nucleophilic displacement by an amine group. Any such leaving group can be used here provided it is readily removed by nucleophilic displacement by amine. Suitable leaving groups are conjugate bases of acids having a pKa of less than about 5. Non-limiting examples of leaving groups useful in the invention include halo, such as bromo, chloro, iodo, O-tosylate (OTos), O-mesylate (OMes), O-triflate (OTf) or O-trimethylsilyl (OTMS).

The nucleophilic substitution reactions may be performed in the presence of a base, for example where the leaving group is a conventional anionic leaving group. The present inventors have found that the yields of cyclised peptide ligands can be greatly increased by suitable choice of solvent and base (and pH) for the nucleophilic substitution reaction, and furthermore that the preferred solvent and base are different from the prior art solvent and base combinations that involve only the formation of thioether linkages. In particular, the present inventors have found that improved yields are achieved when using a trialkylamine base, i.e. a base of formula NR₁R₂R₃, wherein R₁, R₂ and R₃ are independently C1-C5 alkyl groups, suitably C2-C4 alkyl groups, in particular C2-C3 alkyl groups. Especially suitable bases are triethylamine and diisopropylethylamine (DIPEA). These bases have the property of being only weakly nucleophilic, and it is thought that this property accounts for the fewer side reactions and higher yields observed with these bases. The present inventors have further found that the preferred solvents for the nucleophilic substitution reaction are polar and protic solvents, in particular MeCN/H₂O containing MeCN and H₂O in volumetric ratios from 1:10 to 10:1, suitably from 2:10 to 10:2 and more suitably from 3:10 to 10:3, in particular from 4:10 to 10:4.

Additional binding or functional activities may be attached to the N or C terminus of the peptide covalently linked to a molecular scaffold. The functional group is, for example, selected from the group consisting of: a group capable of binding to a molecule which extends the half-life of the peptide ligand in vivo, and a molecule which extends the half-life of the peptide ligand in vivo. Such a molecule can be, for instance, HSA or a cell matrix protein, and the group capable of binding to a molecule which extends the half-life of the peptide ligand in vivo is an antibody or antibody fragment specific for HSA or a cell matrix protein. Such a molecule may also be a conjugate with high molecular weight PEGs.

In one embodiment, the functional group is a binding molecule, selected from the group consisting of a second peptide ligand comprising a peptide covalently linked to a molecular scaffold, and an antibody or antibody fragment. 2, 3, 4, 5 or more peptide ligands may be joined together. The specificities of any two or more of these derivatives may be the same or different; if they are the same, a multivalent binding structure will be formed, which has increased avidity for the target compared to univalent binding molecules. The molecular scaffolds, moreover, may be the same or different, and may subtend the same or different numbers of loops.

The functional group can moreover be an effector group, for example an antibody Fc region.

Attachments to the N or C terminus may be made prior to binding of the peptide to a molecular scaffold, or afterwards. Thus, the peptide may be produced (synthetically, or by biologically derived expression systems) with an N or C terminal peptide group already in place. Preferably, however, the addition to the N or C terminus takes place after the peptide has been combined with the molecular backbone to form a conjugate. For example, Fluorenylmethyloxycarbonyl chloride can be used to introduce the Fmoc protective group at the N-terminus of the peptide. Fmoc binds to serum albumins including HSA with high affinity, and Fmoc-Trp or Fmoc-Lys bind with an increased affinity. The peptide can be synthesised with the Fmoc protecting group left on, and then coupled with the scaffold through the alkylaminos. An alternative is the palmitoyl moiety which also binds HSA and has, for example been used in Liraglutide to extend the half-life of this GLP-1 analogue.

Alternatively, a conjugate of the peptide with the scaffold can be made, and then modified at the N-terminus, for example with the amine- and sulfhydryl-reactive linker N-e-maleimidocaproyloxy) succinimide ester (EMCS). Via this linker the peptide conjugate can be linked to other peptides, for example an antibody Fc fragment.

The binding function may be another peptide bound to a molecular scaffold, creating a multimer; another binding protein, including an antibody or antibody fragment; or any other desired entity, including serum albumin or an effector group, such as an antibody Fc region.

Additional binding or functional activities can moreover be bound directly to the molecular scaffold. In embodiments, the scaffold may further comprise a reactive group to which the additional activities can be bound. Preferably, this group is orthogonal with respect to the other reactive groups on the molecular scaffold, to avoid interaction with the peptide. In one embodiment, the reactive group may be protected, and deprotected when necessary to conjugate the additional activities.

Accordingly, in a further aspect of the invention, there is provided a drug conjugate comprising a peptide ligand as defined herein conjugated to one or more effector and/or functional groups.

Effector and/or functional groups can be attached, for example, to the N or C termini of the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains. An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold.

Peptides such as those derived from such as VP22, HIV-Tat, a homeobox protein of Drosophila (Antennapedia), e.g. as described in Chen and Harrison, Biochemical Society Transactions (2007) Volume 35, part 4, p821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p10444), the 18 amino acid ‘model amphipathic peptide’ (Oehlke et al (1998) Biochim Biophys Acts Volume 1414 p127) and arginine rich regions of the HIV TAT protein. Non peptidic approaches include the use of small molecule mimics or SMOCs that can be easily attached to biomolecules (Okuyama et al (2007) Nature Methods Volume 4 p153). Other chemical strategies to add guanidinium groups to molecules also enhance cell penetration (Elson-Scwab et al (2007) J Biol Chem Volume 282 p13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

RGD peptides, which bind to integrins which are present on many cells, may also be incorporated.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half life in the range 12 to 60 hours. In a further embodiment, it will have a tβ half-life of a day or more. In a further embodiment still, it will be in the range 12 to 26 hours.

In one particular embodiment of the invention, the functional group conjugated to the looped peptide is selected from a metal chelator, which is suitable for complexing metal radioisotopes of medicinal relevance. Such effectors, when complexed with said radioisotopes, can present useful agents for cancer therapy. Suitable examples include DOTA, NOTA, EDTA, DTPA, HEHA, SarAr and others (Targeted Radionuclide therapy, Tod Speer, Wolters/Kluver Lippincott Williams & Wilkins, 2011).

Possible effector groups also include enzymes, for instance such as carboxypeptidase G2 for use in enzyme/prodrug therapy, where the peptide ligand replaces antibodies in ADEPT.

In one particular embodiment of this aspect of the invention, the functional group is selected from a drug, such as a cytotoxic agent for cancer therapy. Suitable examples include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin and others.

In one further particular embodiment of the invention according to this aspect, the cytotoxic agent is selected from DM1 or MMAE.

DM1 is a cytotoxic agent which is a thiol-containing derivative of maytansine and has the following structure:

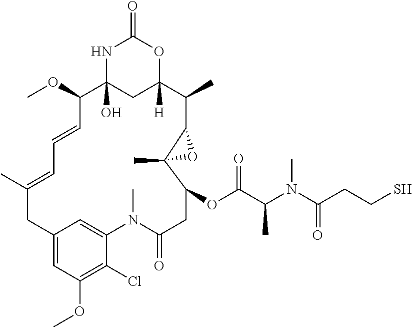 [SMILES: COc1cc2cc(c1Cl)N(C)C(=O)C[C@H](OC(=O)[C@H](C)N(C)C(=O)CCS)[C@@]1(C)O[C@H]1[C@H](C)[C@@H]1C[C@@](O)(NC(=O)O1)[C@H](OC)/C=C/C=C(\C)C2]

Monomethyl auristatin E (MMAE) is a synthetic antineoplastic agent and has the following structure:

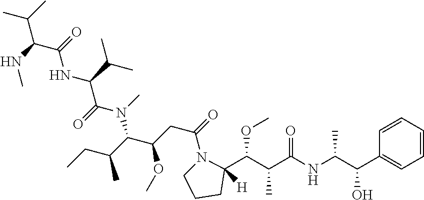 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In one embodiment, the cytotoxic agent is linked to the bicyclic peptide by a cleavable bond, such as a disulphide bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

Thus, in one embodiment, the cytotoxic agent is selected from a compound of formula:

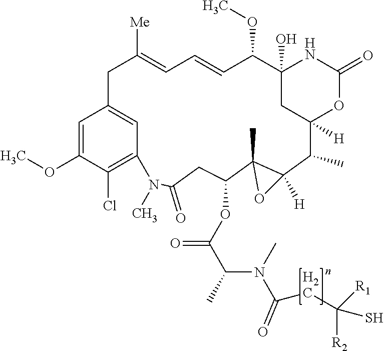

wherein n represents an integer selected from 1 to 10; and
R₁ and R₂ independently represent hydrogen or methyl groups.

In one embodiment of the compound of the above formula, n represents 1 and R₁ and R₂ both represent hydrogen (i.e. the maytansine derivative DM1).

In an alternative embodiment of the compound of the above formula, n represents 2, R₁ represents hydrogen and R₂ represents a methyl group (i.e. the maytansine derivative DM3).

In one embodiment of the compound, n represents 2 and R₁ and R₂ both represent methyl groups (i.e. the maytansine derivative DM4).

It will be appreciated that the cytotoxic agent can form a disulphide bond, and in a conjugate structure with a bicyclic peptide, the disulphide connectivity between the thiol-toxin and thiol-bicycle peptide is introduced through several possible synthetic schemes.

In one embodiment, the bicyclic peptide component of the conjugate has the following structure:

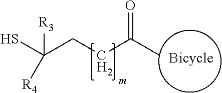

wherein m represents an integer selected from 0 to 10,
Bicycle represents any suitable looped peptide structure as described herein; and
R₃ and R₄ independently represent hydrogen or methyl.

Compounds of the above formula where R₃ and R₄ are both hydrogen are considered unhindered and compounds of the above formula where one or all of R₃ and R₄ represent methyl are considered hindered.

It will be appreciated that the bicyclic peptide of the above formula can form a disulphide bond, and in a conjugate structure with a cytotoxic agent described above, the disulphide connectivity between the thiol-toxin and thiol-bicycle peptide is introduced through several possible synthetic schemes.

In one embodiment, the cytotoxic agent is linked to the bicyclic peptide by the following linker:

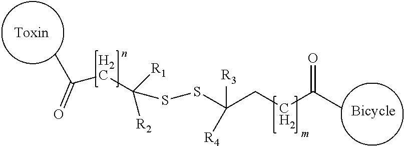

wherein R₁, R₂, R₃ and R₄ represent hydrogen or C1-C6 alkyl groups;
Toxin refers to any suitable cytotoxic agent defined herein;
Bicycle represents any suitable looped peptide structure as described herein;
n represents an integer selected from 1 to 10; and
m represents an integer selected from 0 to 10.

When R₁, R₂, R₃ and R₄ are each hydrogen, the disulphide bond is least hindered and most susceptible to reduction. When R₁, R₂, R₃ and R₄ are each alkyl, the disulphide bond is most hindered and least susceptible to reduction. Partial substitutions of hydrogen and alkyl yield a gradual increase in resistance to reduction, and concomitant cleavage and release of toxin. Preferred embodiments include: R₁, R₂, R₃ and R₄ all H; R₁, R₂, R₃ all H and R₄=methyl; R₁, R₂=methyl and R₃, R₄=H; R₁, R₃=methyl and R₂, R₄=H; and R₁, R₂=H, R₃, R₄=C1-C6 alkyl.

In one embodiment, the toxin of compound is a maytansine and the conjugate comprises a compound of the following formula:

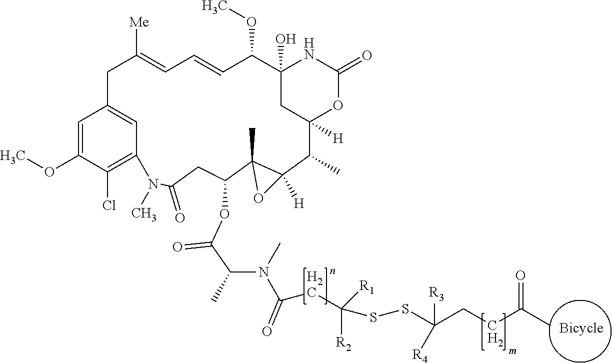

wherein R₁, R₂, R₃ and R₄ are as defined above;
Bicycle represents any suitable looped peptide structure as defined herein;
n represents an integer selected from 1 to 10; and
m represents an integer selected from 0 to 10.

Further details and methods of preparing the above-described conjugates of bicycle peptide ligands with toxins are described in detail in our published patent application WO2016/067035 and pending application GB1607827.1 filed on 4 May 2016. The entire disclosure of these applications is expressly incorporated herein by reference.

The linker between the toxin and the bicycle peptide may comprise a triazole group formed by click-reaction between an azide-functionalized toxin and an alkyne-functionalized bicycle peptide structure (or vice-versa). In other embodiments, the bicycle peptide may contain an amide linkage formed by reaction between a carboxylate-functionalized toxin and the N-terminal amino group of the bicycle peptide.

The linker between the toxin and the bicycle peptide may comprise a cathepsin-cleavable group to provide selective release of the toxin within the target cells. A suitable cathepsin-cleavable group is valine-citrulline.

The linker between the toxin and the bicycle peptide may comprise one or more spacer groups to provide the desired functionality, e.g. binding affinity or cathepsin cleavability, to the conjugate. A suitable spacer group is para-amino benzyl carbamate (PABC) which may be located intermediate the valine-citrulline group and the toxin moiety.

Thus, in embodiments, the bicycle peptide-drug conjugate may have the following structure made up of Toxin-PABC-cit-val-triazole-Bicycle:

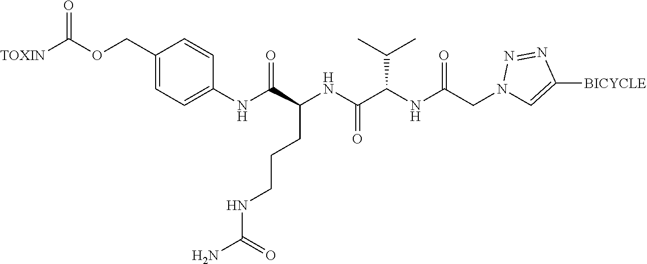

In further embodiments, the bicycle peptide-drug conjugate may have the following structure made up of Toxin-PABC-cit-val-dicarboxylate-Bicycle:

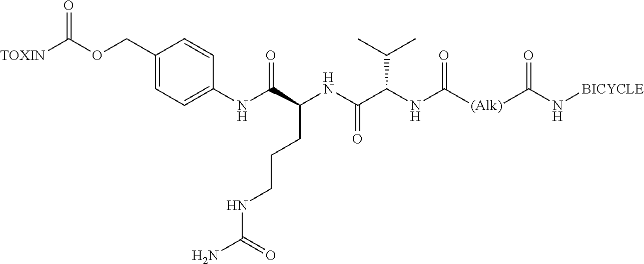

wherein (alk) is an alkylene group of formula CₙH₂ₙ wherein n is from 1 to 10 and may be linear or branched, suitably (alk) is n-propylene or n-butylene.

Suitably, the bicycle peptide-drug conjugate is selected from the group consisting of BT17BDC53, BT17BDC59, BT17BDC61, BT17BDC62, and BT17BDC68, as defined hereinbelow.

Peptide ligands according to the present invention may be employed in in vivo therapeutic and prophylactic applications, in vitro and in vivo diagnostic applications, in vitro assay and reagent applications, and the like.

In general, the use of a peptide ligand can replace that of an antibody. Derivatives selected according to the invention are of use diagnostically in Western analysis and in situ protein detection by standard immunohistochemical procedures; for use in these applications, the derivatives of a selected repertoire may be labelled in accordance with techniques known in the art. In addition, such peptide ligands may be used preparatively in affinity chromatography procedures, when complexed to a chromatographic support, such as a resin. All such techniques are well known to one of skill in the art. Peptide ligands according to the present invention possess binding capabilities similar to those of antibodies, and may replace antibodies in such assays.

Diagnostic uses include any uses which to which antibodies are normally put, including test-strip assays, laboratory assays and immunodiagnostic assays.

Therapeutic and prophylactic uses of peptide ligands prepared according to the invention involve the administration of derivatives selected according to the invention to a recipient mammal, such as a human. Substantially pure peptide ligands of at least 90 to 95% homogeneity are preferred for administration to a mammal, and 98 to 99% or more homogeneity is most preferred for pharmaceutical uses, especially when the mammal is a human Once purified, partially or to homogeneity as desired, the selected peptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

Generally, the present peptide ligands will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a peptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cyclosporine, methotrexate, adriamycin or cisplatinum, and immunotoxins. Pharmaceutical compositions can include “cocktails” of various cytotoxic or other agents in conjunction with the selected antibodies, receptors or binding proteins thereof of the present invention, or even combinations of selected peptides according to the present invention having different specificities, such as peptides selected using different target derivatives, whether or not they are pooled prior to administration.

The route of administration of pharmaceutical compositions according to the invention may be any of those commonly known to those of ordinary skill in the art. For therapy, including without limitation immunotherapy, the selected antibodies, receptors or binding proteins thereof of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counter-indications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that use levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the selected repertoires of peptides described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

The invention is further described with reference to the following examples.

### EXAMPLES

### Materials and Methods

### Protein Expression

The MT1-MMP hemopexin-like repeats (also known as the MT1-MMP hemopexin domain), residues Cys319-Gly511 from the human gene, were transiently expressed in HEK293 cells as secreted N-terminally His6-tagged soluble protein, using the pEXPR-IBA42 (IBA) expression vector. Following expression, the protein was purified by Nickel-NTA affinity chromatography followed by gel filtration, and purity was checked by SDS-PAGE. Batch to batch variability was also monitored by fluorescence thermal shift experiments in the presence/absence of a hemopexin domain binding bicycle.

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with the following side chain protecting groups: Arg(Pbf); Asn(Trt); Asp(OtBu); Cys(Trt); GIu(OtBu); Gln(Trt); His(Trt); Lys(Boc); Ser(tBu); Thr(tBu); Trp(Boc); and Tyr(tBu) (Sigma). The coupling reagent was HCTU (Pepceuticals), diisopropylethylamine (DIPEA, Sigma) was employed as a base, and deprotection was achieved with 20% piperidine in DMF (AGTC). Syntheses were performed using 0.37 mmol/gr Fmoc-Rink amide AM resin (AGTC), Fmoc-amino acids were utilised at a four-fold excess, and base was at a four-fold excess with respect to the amino acids. Amino acids were dissolved at 0.2M in DMSO, HCTU at 0.4M in DMF, and DIPEA at 1.6M in N-methylpyrrolidone (Alfa Aesar). Conditions were such that coupling reactions contained between 20 to 50% DMSO in DMF, which reduced aggregation and deletions during the solid phase synthesis and enhanced yields. Coupling times were generally 30 minutes, and deprotection times 2×5 minutes. Fmoc-N-methylglycine (Fmoc-Sar-OH, Merck) was coupled for 1 hr, and deprotection and coupling times for the following residue were 20 min and 1 hr, respectively. After synthesis, the resin was washed with dichloromethane, and dried. Cleavage of side-chain protecting groups and from the support was effected using 10 mL of 95:2.5:2.5:2.5 v/v/v/w TFA/H₂O/iPr₃ SiH/dithiothreitol for 3 hours. Following cleavage, the spent resin was removed by filtration, and the filtrate was added to 35 mL of diethylether that had been cooled at −80° C. Peptide pellet was centrifuged, the etheric supernatant discarded, and the peptide pellet washed with cold ether two more times. Peptides were then resolubilised in 5-10 mL acetonitrile-water and lyophilised. A small sample was removed for analysis of purity of the crude product by mass spectrometry (MALDI-TOF, Voyager DE from Applied Biosystems). Following lyophilisation, peptide powders were taken up in 10 mL 6 M guanidinium hydrochloride in H₂O, supplemented with 0.5 mL of 1 M dithiothreitol, and loaded onto a C8 Luna preparative HPLC column (Phenomenex). Solvents (H₂O, acetonitrile) were acidified with 0.1% heptafluorobutyric acid. The gradient ranged from 30-70% acetonitrile in 15 minutes, at a flowrate of 15-20 mL/min, using a Gilson preparative HPLC system. Fractions containing pure linear peptide material (as identified by MALDI) were used for preparation of the bicycle derivatives by coupling to a scaffold molecule as described further below.

All amino acids, unless noted otherwise, were used in the L-configurations. Non-natural amino acids were incorporated into peptide sequence using the general methods described above. The list of non-natural amino acid precursors employed herein are summarised in the table below:

| | Short | |
|---|---|---|
| Supplier | name | Full chemical name |
| AGTC | D-Asp | Fmoc-D-Asp(tBu)-OH |
| Iris Biotech | HPhe | Fmoc-L-Homophenylalanine |
| Alfa Aesar | 5FPhe | Fmoc-pentafluoro-L-phenylalanine |
| PolyPeptide Gropu | 4BrPhe | Fmoc-4-bromo-L-phenylalanine |
| Iris Biotech | bAla | Fmoc-beta-Ala-OH |
| Iris Biotech | 3Pal | Fmoc-L-3Pal-OH |
| Iris Biotech | 4Pal | Fmoc-L-4Pal-OH |
| Iris Biotech | D-Pro | Fmoc-D-Pro-OH |
| Merck Novabiochem | Aib | Fmoc-Aib-OH |
| Merck Novabiochem | D-Ala | Fmoc-D-Ala-OH |
| Merck Novabiochem | D-Arg | Fmoc-D-Arg(Pbf)-OH |
| Merck Novabiochem | D-Gln | Fmoc-D-Gln(Trt)-OH |
| Merck Novabiochem | D-His | Fmoc-D-His(Trt)-OH |
| Merck Novabiochem | Hyp | Fmoc-Hyp(tBu)-OH |
| Merck Novabiochem | D-Leu | Fmoc-D-Leu-OH |
| Merck Novabiochem | HArg | Fmoc-L-HArg(Boc)2-0H |
| Peptech Corporation | 4,4-BPA1 | Fmoc-L-4,4’-Biphenylalanine |
| Peptech Corporation | 3,3-DPA | Fmoc-L-3,3’-Diphenylalanine |
| Peptech Corporation | Dpg | Fmoc-Dipropylglycine |
| Peptech Corporation | 1Nal | Fmoc-L-1-Naphthylalanine |
| Peptech Corporation | 2NA1 | Fmoc-L-2-Naphthylalanine |
| Peptech Corporation | Pip | Fmoc-L-Pipecolic acid |
| Polypeptide Group | Aze | Fmoc-L-azetidine-2-carboxylic acid |
| Polypeptide Group | Cha | Fmoc-beta-cyclohexyl-L-alanine |
| Polypeptide Group | 4FluoPro | (2S,4R)-Fmoc-4-fluoro-pyrrolidine- |
| | | 2-carboxylic acid |
| AGTC | D-Asp | Fmoc-D-Asp(tBu)-OH |
| Merck | tBuGly | Fmoc-α-tert-butylglycine |
| Iris Biotech | Chg | Fmoc-L-cyclohexylglycine |
| Fluorochem | Phg | Fmoc-Phenylglycine-OH |
| Iris Biotech | 3Pal | Fmoc-L-3Pal-OH |
| Iris Biotech | 4Pal | Fmoc-L-4Pal-OH |
| Merck Novabiochem | D-Leu | Fmoc-D-Leu-OH |
| Merck Novabiochem | HArg | Fmoc-L-HArg(Boc)2-OH |
| Polypeptide Group | 3,4 | Fmoc-3,4-dichloro-L-phenylalanine |
| | DCPhe | |
| Polypeptide Group | Cha | Fmoc-beta-cyclohexyl-L-alanine |

In addition, the following non-natural amino acid precursors were used for the preparation of the DAP and N-MeDAP modified peptides:

| Compound | CAS | Mw | Supplier |
|---|---|---|---|
| Fmoc-L-Dap | 446847-80-9 | 440.49 | Iris Biotech |
| (Boc,Me)-OH | | | GMBH |
| Fmoc-Dap(Boc)-OH | 162558-25-0 | 426.46 | Sigma Aldrich |

### Binding Affinity to MT1-MMP

Binding affinity was measured using competition assays using Fluorescence Polarisation (anisotropy).

Fluorescent tracers referred to herein are bicyclic peptides that have been fluoresceinated using 5,6-carboxyfluorescein. Fluoresceination may be performed on the N-terminal amino group of the peptide, which is separated from the bicycle core sequence by a sarcosine spacer (usually Sar5). This can be done during Fmoc solid phase synthesis or post-synthetically (after cyclisation with TBMB and purification) if the N-terminal amino group is unique to the peptide. Fluoresceination can also be performed on the C-terminus, usually on a Lysine introduced as the first C-terminal residue, which is then separated from the bicycle core sequence by a sarcosine spacer (usually Sar6). Thus, N-terminal tracers can have a molecular format described as Fluo-Gly-Sar5-A(BicycleCoreSequence), and (BicycleCoreSequence)-A-Sar6-K(Fluo) for a C-terminally fluoresceinated construct. Fluorescent tracers used in the Examples are A-(17-69)-A-Sar6-K(Fluo), A-(17-69-07)-A-Sar6-K(Fluo), and A-(17-69-12)-A-Sar6-K(Fluo). Due to the acidic nature of the 17-69 fluorescent peptides, they were typically prepared as concentrated DMSO stocks, from which dilution were prepared in 100 mM Tris pH 8 buffer.

Due to their high affinities to the MT1-MMP Hemopexin domain (PEX), the fluoresceinated derivatives herein can be used for competition experiments (using FP for detection). Here, a pre-formed complex of PEX with the fluorescent PEX-binding tracer is titrated with free, non-fluoresceinated bicyclic peptide. Since all 17-69-based peptides are expected to bind at the same site, the titrant will displace the fluorescent tracer from PEX. Dissociation of the complex can be measured quantitatively, and the Kd of the competitor (titrant) to the target protein determined. The advantage of the competition method is that the affinities of non-fluoresceinated bicyclic peptides can be determined accurately and rapidly.

Concentrations of tracer are usually at the Kd or below (here, 1 nM), and the binding protein (here, hemopexin of MT1-MMP) is at a 15-fold excess such that >90% of the tracer is bound. Subsequently, the non-fluorescent competitor bicyclic peptide (usually just the bicycle core sequence) is titrated, such that it displaces the fluorescent tracer from the target protein. The displacement of the tracer is measured and associated with a drop in fluorescence polarisation. The drop in fluorescence polarisation is proportional to the fraction of target protein bound with the non-fluorescent titrant, and thus is a measure of the affinity of titrant to target protein.

The raw data is fit to the analytical solution of the cubic equation that describes the equilibria between fluorescent tracer, titrant, and binding protein. The fit requires the value of the affinity of fluorescent tracer to the target protein, which can be determined separately by direct binding FP experiments (see previous section). The curve fitting was performed using Sigmaplot 12.0 and used an adapted version of the equation described by Zhi-Xin Wang (FEBS Letters 360 (1995) 111-114).

### Reference Example 1

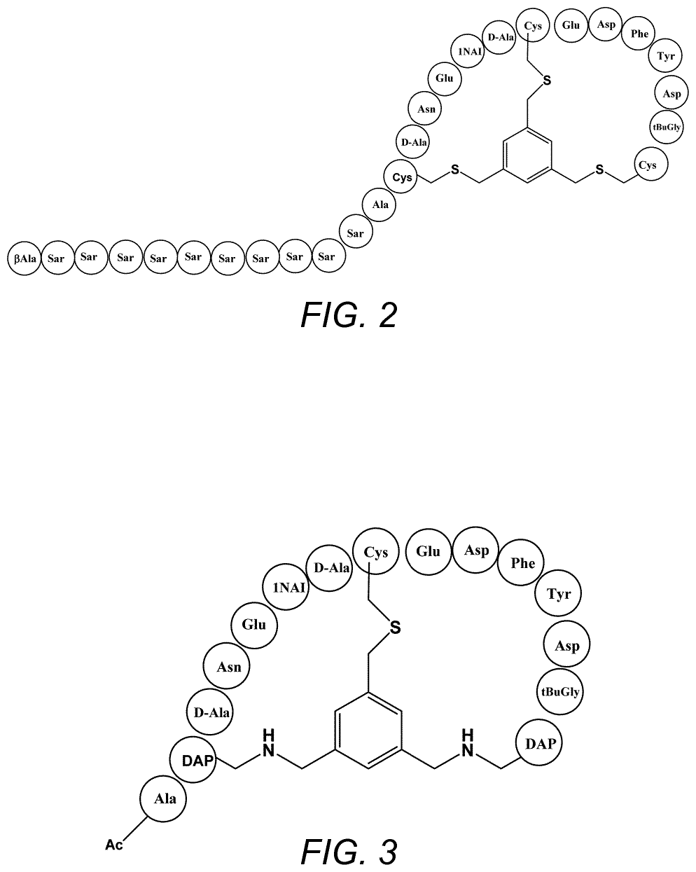

The Bicyclic Peptide chosen for comparison of thioether to alkylamino scaffold linkage was designated 17-69-07-N241. It is a bicycle conjugate of a thioether-forming peptide with a trimethylene benzene scaffold. The structure of this bicycle derivative is shown schematically in FIG. 2. The linear peptide before conjugation has sequence:

### H-(β-Ala)-Sar10-Ala-Cys-(D-Ala)-Asn-Glu-(1Nal)-(D-Ala)-Cys-Glu-Asp-Phe-Tyr-Asp-(tBuGly)-Cys-NH₂

Conjugation to 1,3,5-tris(bromomethyl)benzene (TBMB, Sigma) was carried out as follows. The linear peptide was diluted with H₂O up to ˜35 mL, ˜500 μL of 100 mM TBMB in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Following lyophilisation, the modified peptide was purified as above, while replacing the Luna C8 with a Gemini C18 column (Phenomenex), and changing the acid to 0.1% trifluoroacetic acid. Pure fractions containing the correct TMB-modified material were pooled, lyophilised and kept at −20° C. for storage.

The resulting Bicycle derivative designated 17-69-07-N241 showed high affinity to MT1-MMP. The measured affinity (Kd) to MT1-MMP of the derivative was 0.23 nM. The derivative is therefore regarded as a promising candidate for targeting tumor cells that express the cell surface metalloproteinase MT1-MMP.

### Example 1

A bicycle peptide designated 17-69-07-N385 was made corresponding to the bicycle region of the peptide ligand of Reference Example 1, minus the b-Ala-Sar10 tail, and with replacement of the first and third cysteine residues by DAP residues forming alkylamino linkages to the TBMB scaffold. The structure of this derivative is shown schematically in FIG. 3.

The linear peptide used to form this bicycle was as follows:

Ac-A(Dap)(D-Ala)NE(1Nal)(D-Ala)CEDFYD(tBuGly)(Dap)

The linear peptide and the bicycle peptide had the following LCMS Characteristics:

| | Retention | m/z |
|---|---|---|
| | Time | present |
| Linear peptide | 4.17 min | 886.1, 1771.7 |
| Cyclised peptide | 4.39 min | 942.7 |

Various reagents for the cyclisation step were tried as follows. Reagents were made up to the concentrations indicated in the table below in the chosen solvent. To a volume of peptide solution was added half that volume of TBMB solution, the mixture stirred well then half of the volume of base solution. The reaction was mixed and sampled periodically for LCMS analysis.

| | | | Volume | | |
|---|---|---|---|---|---|
| | Initial | | equivalents | Final | |
| | Solution | | added to | Reaction | |
| Reagent | Concentration | | reaction | Concentration | |
| Peptide | 1.0 | mM | 1.0 | 0.5 | mM |
| TBMB | 2.6 | mM | 0.5 | 0.65 | mM |
| Base | 200 | mM | 0.5 | 50 | mM |

Example: to 50 μL peptide solution was added 25 μL TBMB solution. The solution was mixed thoroughly then 25 μL base solution was added.

In cases where the solvent used is DMF, all reagents are made up in DMF. In cases where the solvent used is DMSO, all reagents are made up in DMSO. In cases where the solvent used is MeCN/H₂O, peptide solutions are made up in 50% MeCN/H₂O, TBMB solutions are made up in MeCN and base solutions are made up in H₂O, except when the base is DIPEA, in which case the base solution is made up in MeCN. All cyclisations were performed at room temperature. The results were as follows (range of spectrum set at 3.5-5.5 min. Spectrum at 220 nm integrated and sum of major peaks taken):

| | | Total | Product | % | | |
|---|---|---|---|---|---|---|
| Solvent | Base | integration | integration | Product | Time | |
| MeCN/H₂O | NEt₃ | 4641.0 | 2857.1 | 62% | 5 | h |
| | Na₂CO₃ | 5470.8 | 546.1 | 10% | 5 | h |
| | NaHCO₃ | 9956.5 | 3530.4 | 35% | 4 | h |
| | NH₄HCO₃ | 6948.9 | 0 | 0% | 5 | h |
| | Tetramethyl | 12130.5 | 211.9 | 2% | 5 | h |
| | guanidine | | | | | |
| | DIPEA | 9081.1 | 5951.6 | 66% | 16 | h |

It can be seen that the purity following cyclisation is highly dependent on the choice of base. Product purity ranges from 2 to 66%, with the latter involving a mixture of Acetonitrile/water in the presence of DIPEA. Unlike the cyclisation of Reference Example 1, the yield is relatively low when using the conventional NaHCO₃ as the base. Best yields are achieved using the trialkylamines, namely triethylamine and diisopropylethylamine (DIPEA).

Comparative binding to MT1-MMP data are shown in FIG. 7. The measured Kd is 0.45 nM, which demonstrates that the change to alkylamino linkages in this example resulted in remarkably little change in binding affinity relative to the thioether linked derivative of Reference Example 1.

### Example 2

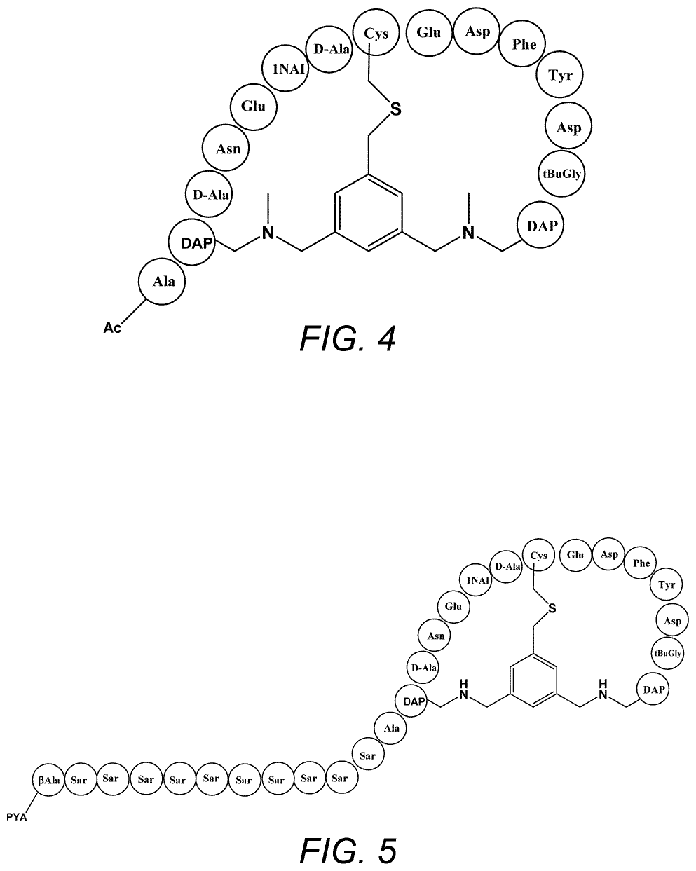

A bicycle peptide designated 17-69-07-N426 was made corresponding to the bicycle peptide of Example 1 with replacement of the DAP residues by N-MeDAP residues. The structure of this derivative is shown schematically in FIG. 4. The linear peptide used to form this bicycle was as follows:

Ac-A(Dap(Me))(D-Ala)NE(1Nal)(D-Ala)CEDFYD(tBuGly)(Dap(Me))

The linear peptide and the bicycle peptide had the following LCMS Characteristics:

| | Retention Time | m/z present |
|---|---|---|
| Linear peptide | 4.19 min | 900.1, 1799.1 |
| Cyclised peptide | 4.38 min | 956.0, 1913.7 |

Various different reaction conditions, solvents, and bases were used for the cyclisation step as described in Example 1, with the following results (all cyclisations performed at room temperature):

| | | Total | Product | % | | |
|---|---|---|---|---|---|---|
| Solvent | Base | integration | integration | Product | Time | |
| MeCN/H₂O | Na₂CO₃ | 21659.8 | 12714.5 | 59% | 1 | h |
| MeCN/H₂O | DIPEA | 10547.7 | 9841.3 | 93% | 16 | h |

The purity following cyclisation is again dependent on the nature of the base. Purity with Na₂CO₃ as base is, as expected, low (see Example 1). Using the optimal condition of Acetonitrile/water in the presence of DIPEA, purity following cyclisation is very high (93%) demonstrating that N-methylation of Dap reduces the level of side reactions.

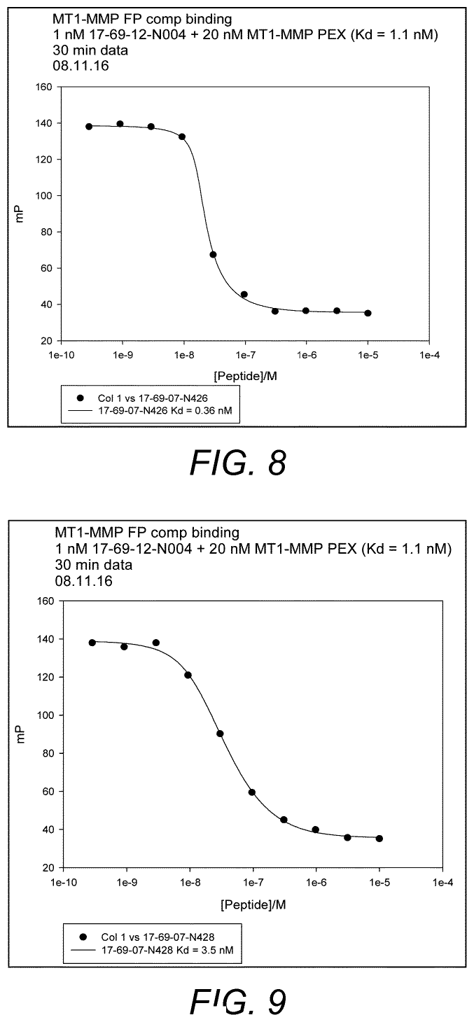

Comparative binding to MT1-MMP data are shown in FIG. 8. The measured Kd is 0.36 nM, which is almost unchanged from the thioether linked derivative of Reference Example 1. This potency is retained despite the two N-methylations on the linkage, and thus the derivative of the present example is of great interest.

### Example 3

A bicycle peptide designated 17-69-07-N428 was made corresponding to the bicycle peptide of Example 1 with replacement of the Tyr9 by Phe9 (removal of Tyr hydroxyl). The linear peptide used to form this bicycle was as follows:

Ac-A(Dap)(D-Ala)NE(1Nal)(D-Ala)CEDFF₉D(tBuGly)(Dap)

The linear peptide and the bicycle peptide had the following LCMS Characteristics:

| | Retention Time | m/z present |
|---|---|---|
| Linear peptide | 4.51 min | 876.5, 1755.4 |
| Cyclised peptide | 4.80 min | 935.3 |

Various different reaction conditions, solvents, and bases were used for the cyclisation step as described in Example 1, with the following results (LCMS range of spectrum set at 4-6 min. Spectrum at 220 nm integrated and sum of major peaks taken):

| | | | Total | Product | % | |
|---|---|---|---|---|---|---|
| Solvent | Base | Temp | integration | integration | Product | Time |
| MeCN/H₂O | DIPEA | rt | 5962.8 | 4209.3 | 71% | 6 h |
| MeCN/H₂O | DIPEA | 50° C. | 5936.4 | 2898.3 | 49% | 1 h |
| DMF | DIPEA | rt | 4804.6 | 84.7 | 2% | 1 h |
| DMF | DIPEA | 50° C. | 4384.8 | 309.7 | 7% | 1 h |
| MeCN/H₂O | TMG | rt | 5050.8 | 2023.8 | 40% | 1 h |
| MeCN/H₂O | K₂CO₃ | rt | 6366.7 | 4109.2 | 65% | 4 h |
| MeCN/H₂O | K₂CO₃ | 50° C. | 5314.5 | 2306.7 | 43% | 1 h |

It can be seen that product purity ranges from 2 to 71%, with the latter involving a mixture of Acetonitrile/water in the presence of DIPEA. The removal of the Tyr-OH (Tyr→Phe9) increases product yield significantly relative to the same reaction with the tyrosine-containing peptide under MeCN/H₂O/TMG/rt or MeCN/H₂O/K₂CO₃/rt conditions. Use of DMSO as solvent gave very messy chromatograms with multiple peaks which could not be easily analysed.

Comparative binding data of this 17-69-07-N428 derivative to MT1-MMP are shown in FIG. 9. The measured Kd is 3.5 nM. The slight reduction in binding affinity relative to the thioether linked derivative of Reference Example 1 would appear to be mainly attributable to the replacement of the Tyr9 by Phe9.

### Example 4

A bicycle peptide designated 17-69-07-N434 was made corresponding to the bicycle peptide of Example 1 with an N-terminal Sar10 spacer similar to that of Reference Example 1, and conjugating group PYA (4-pentynoic acid, for “click” derivatisation with toxin). The structure of this derivative is shown schematically in FIG. 5. The linear peptide used to form this bicycle was as follows:

(PYA)-(B-Ala)-Sar10-A(Dap)(D-Ala)NE(1Nal)(D-Ala)CEDFYD(tBuGly)(Dap)

The linear peptide and the bicycle peptide had the following LCMS Characteristics:

| | Retention Time | m/z present |
|---|---|---|
| Linear peptide | 4.18 | 863.7, 1294.8 |
| Cyclised peptide | 4.37 | 902.6, 1353.0 |

Cyclisation was performed as follows:

| | | | Total | Product | % | |
|---|---|---|---|---|---|---|
| Solvent | Base | Temp | integration | integration | Product | Time |
| MeCN/H₂O | DIPEA | rt | 4445.6 | 2666.5 | 60% | 4 h |

The resulting derivative 17-69-07-N434 is the Dap1/3 equivalent of N241 (Reference Example 1) with an N-terminal alkyne required for derivatisation with effectors, i.e. toxins. This peptide can be cyclised with TBMB at 60% purity. The measured k_{d} with MT1-MMP was 1.52 nM, making this bicycle peptide highly suitable for targeting MT1-MMP.

### Example 5

Replacement of the TBMB scaffold molecule used in Examples 1 to 3 by TBAB was performed as follows.

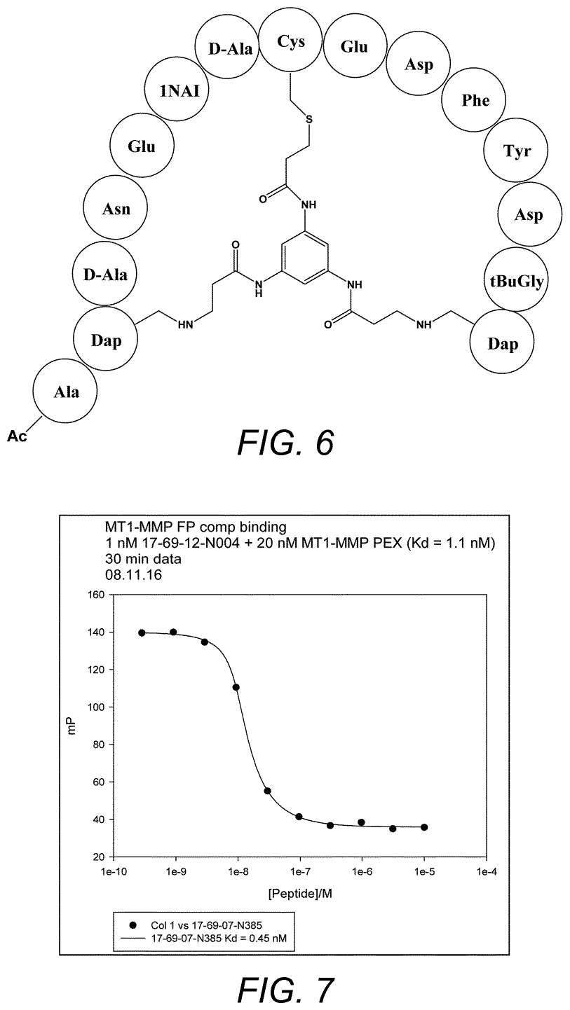

The linear peptides used to form 17-69-07-N385, 17-69-07-N426 and 17-69-07-N428 in Examples 1 to 3 were cyclised with TBAB at the same concentrations and equivalents as those used for TBMB. The structure of the TBAB derivative with the N385 peptide is shown schematically in FIG. 6.

DIPEA was employed as base of choice with a solvent mixture of MeCN/H₂O at room temperature. The following results were achieved.

| | Product | | | | |
|---|---|---|---|---|---|
| | retention | Total | Product | % | |
| Peptide | time | integration | integration | Product | Time |
| 17-69-07-N385 | 4.28 min | 9399.8 | 8830.0 | 94% | 16 h |
| 17-69-07-N426 | 4.47 min | 11941.6 | 11485.1 | 96% | 16 h |
| 17-69-07-N428 | 4.70 min | 8321.8 | 7941.5 | 95% | 16 h |

These results show that TBAB (haloacetyl-) chemistry offers higher cyclisation rates and greater selectivity than TBMB, as can be seen from the % Product column

### Example 6

A bicycle peptide designated 17-69-07-N474 was made corresponding to the bicycle peptide of Example 1 with replacement of the Cys6 by Dap(Me). The linear peptide used to form this bicycle was as follows: Ac-A(Dap(Me))(D-Ala)NE(1Nal)(D-Ala)(Dap(Me))EDFYD(tBuGly)(Dap(Me))

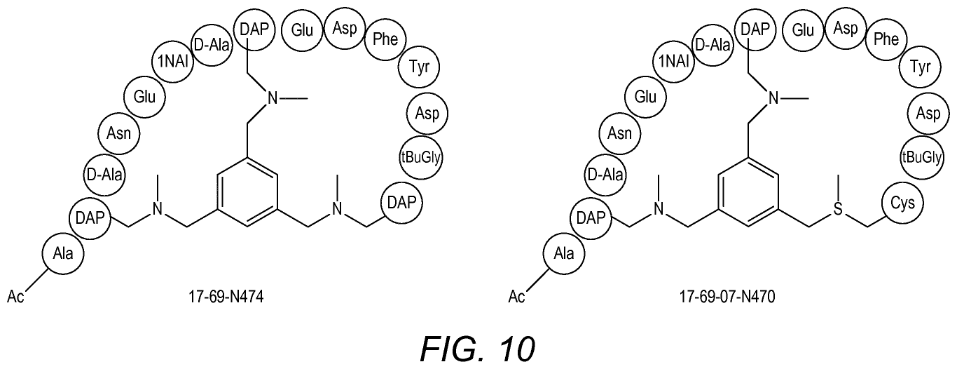

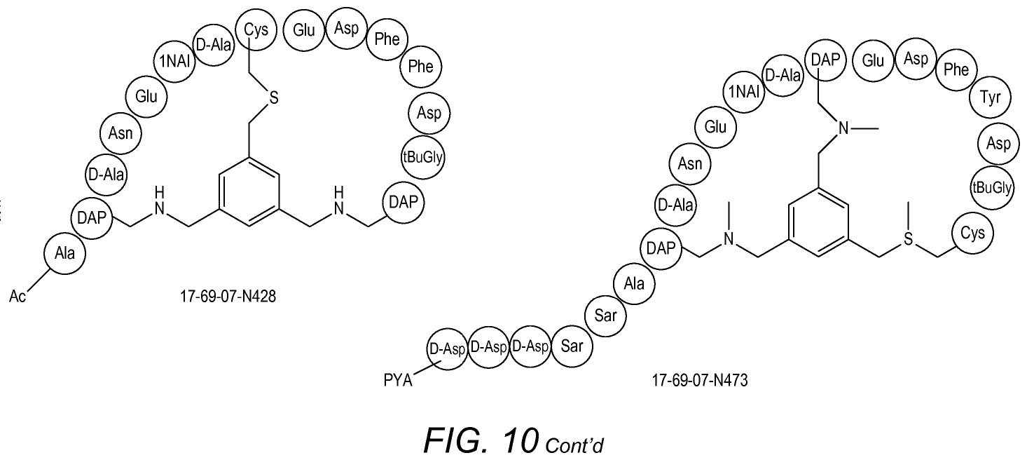

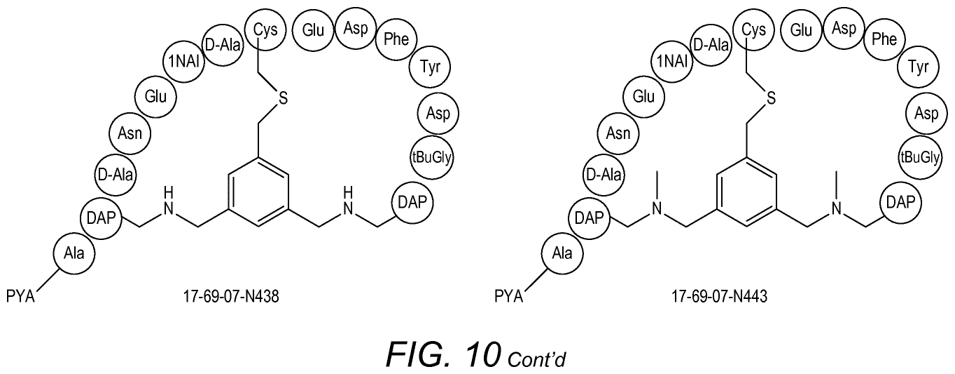

The structure of the TBMB derivative with the N385 peptide is shown schematically in FIG. 10.

The linear peptide and the bicycle peptide had the following LCMS Characteristics:

| | Retention | |
|---|---|---|
| | time | m/z present |
| Linear peptide | 3.61 min | 599.64, 898.96, 1795.90 |
| Cyclised peptide | 3.98 min | 637.68, 956.03, 1910.04 |

The cyclisation was performed according to the following procedure: 50 μL of a 1 mM solution of peptide in MeCN/H₂O (1:1) was mixed with 25 μL 2.6 mM TBMB in MeCN, then 25 μL 200 mM DIPEA in MeCN/H₂O (pH adjusted to 10 with acetic acid) was added and the solution mixed. (1.3 equivalents TBMB and 100 equivalents base with respect to peptide present in the reaction). LCMS samples were taken after 4 hours, and overnight with the reaction proceeding as shown in the table below.

| Total | Product | % | | |
|---|---|---|---|---|
| integration | integration | Product | Time | |
| 3120 | 1248 | 40 | 4 | h |
| 3784 | 3632 | 96 | 18 | h |

(overnight)

Binding to MT1-MMP was assessed in the same manner as the other examples. The measured Kd is 8.0 nM, which is less than the thioether linked derivative of Reference Example 1. This compound still binds with high affinity despite the three N-methylDaps on the linkage, and thus the derivative of the present example is of great interest.

### Example 7

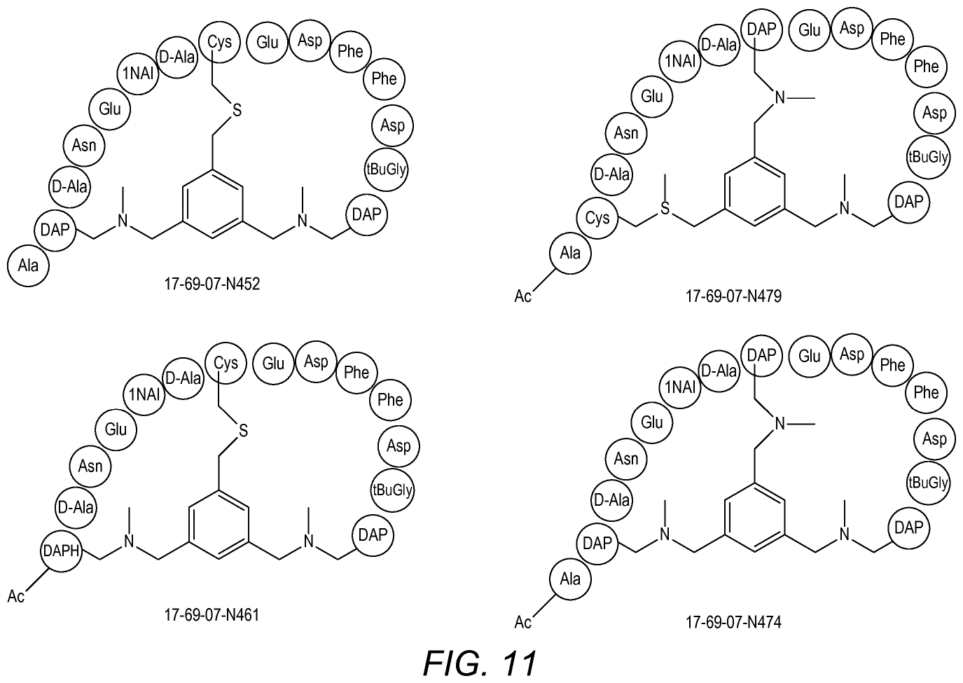

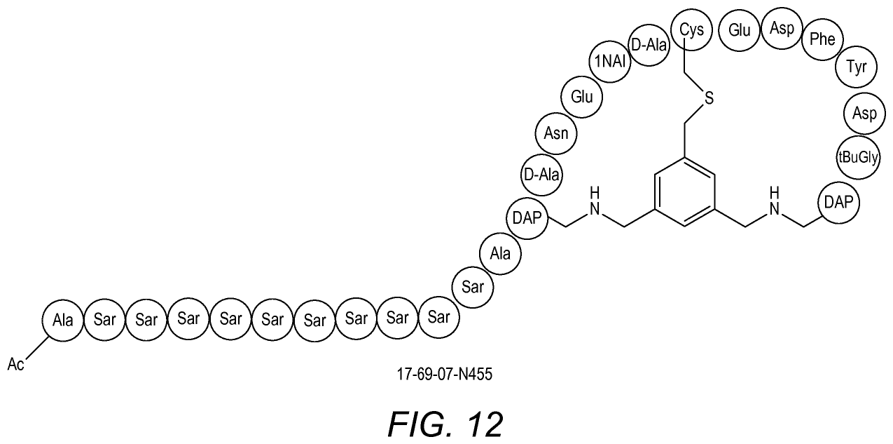

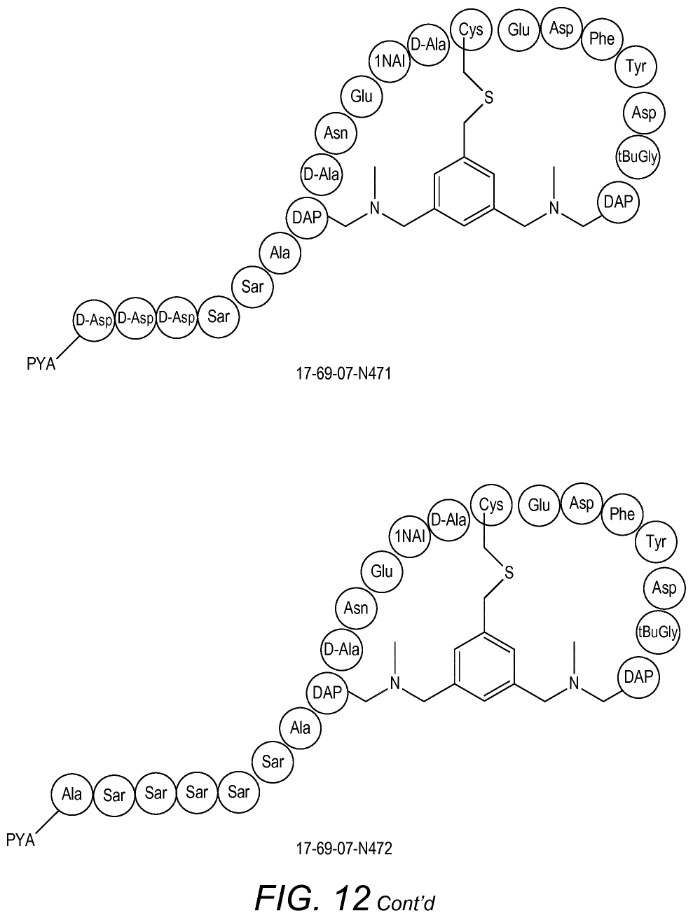

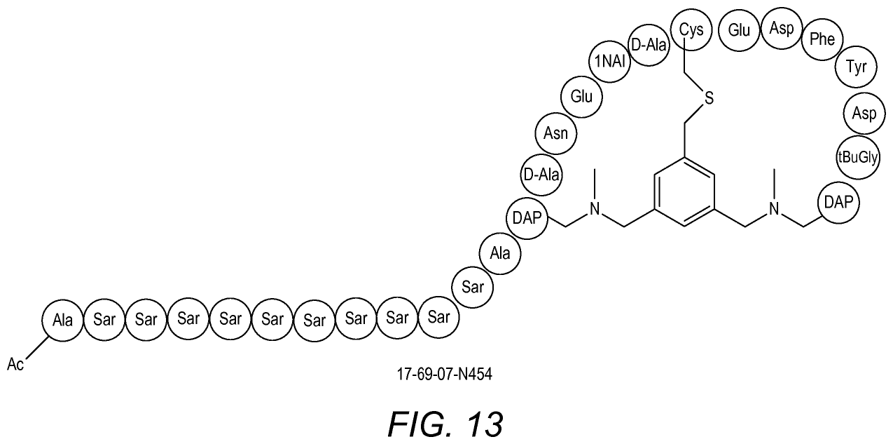

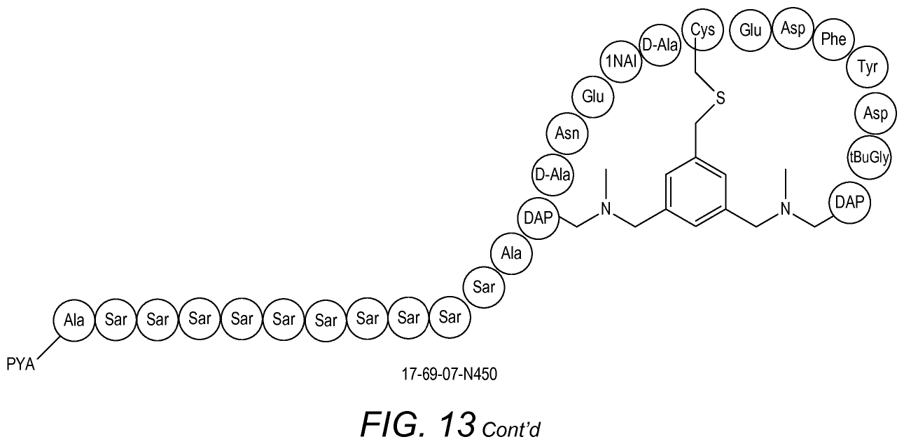

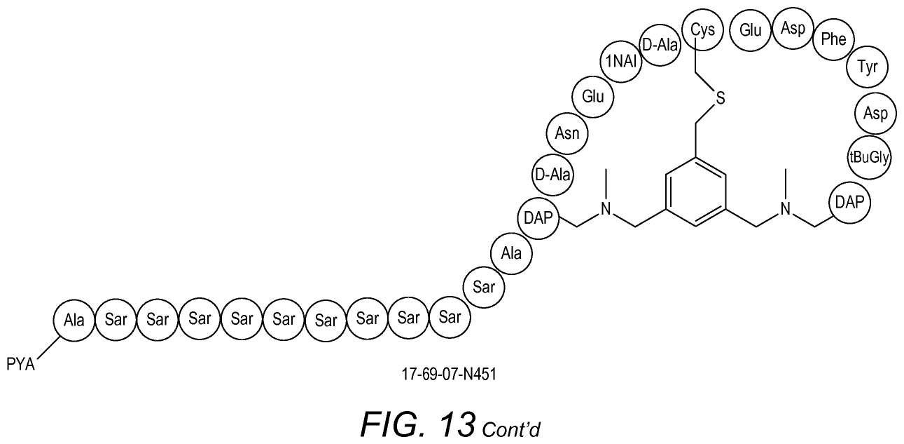

The following further bicycle peptides according to the invention were prepared and tested for binding affinity with MT1-MMP using the methods described above. Schematic structures of these bicycle peptide compounds are shown in FIGS. 10-13.

| | | Binding |
|---|---|---|
| Compound ID | Sequence | Affinity |
| 17-69-07-N428 | Ac-A(Dap)(D-Ala)NE(1Nal)(D-Ala)CEDFFD | n/a |
| | (tBuGly)(Dap) | |
| 17-69-07-N438 | PYA-A(Dap)(D-Ala)NE(1Nal)(D-Ala)CEDFYD | Ki (h) = 3.1 nM |
| | (tBuGly)(Dap) | |
| 17-69-07-N455 | Ac-(B-Ala)-Sar10-A(Dap)(D-Ala)NE(1Nal)(D-Ala) | Ki (h) = 4 nM |
| | CEDFYD(tBuGly)(Dap) | |
| 17-69-07-N470 | Ac-A(Dap(Me))(D-Ala)NE(1Nal)(D- | Ki (h) = 8.3 nM |
| | Ala)(Dap(Me))EDFYD (tBuGly)C | |
| 17-69-07-N473 | (PYA)-(D-Asp)3-Sar2-A(Dap(Me))(D-Ala)NE(1Nal) | Ki (h) = 7.3 nM |
| | (D-Ala) (Dap(Me))EDFYD(tBuGly)C | |
| 17-69-07-N443 | PYA-A(Dap(Me))(D-Ala)NE(1Nal)(D-Ala)CEDFYD | Ki (h) = 3.2 nM |
| | (tBuGly) (Dap(Me)) | |
| 17-69-07-N471 | (PYA)-(D-Asp)3-Sar2-A(Dap(Me))(D-Ala)NE(1Nal) | Ki (h) = 4.6 nM |
| | (D-Ala) CEDFYD(tBuGly)(Dap(Me)) | |
| 17-69-07-N472 | (PYA)-(B-Ala)-Sar5-A(Dap(Me))(D-Ala)NE(1Nal)(D- | Ki (h) = 3.4 nM |
| | Ala) CEDFYD(tBuGly)(Dap(Me)) | |
| 17-69-07-N454 | Ac-(B-Ala)-Sar10-A(Dap(Me))(D-Ala)NE(1Nal)(D- | Ki (h) = 3.0 nM |
| | Ala) CEDFYD (tBuGly)(Dap(Me)) | |
| 17-69-07-N452 | A(Dap(Me))(D-Ala)NE(1Nal)(D-Ala)CEDFYD | Ki (h) = 4.0 nM |
| | (tBuGly)(Dap(Me)) | |
| 17-69-07-N450 | (PYA)-(B-Ala)-Sar10-A(Dap(Me))(D-Ala)NE(1Nal) | Ki = 3.7 nM; |
| | (D-Ala) CEDFYD(tBuGly)(Dap(Me)) | Kd = 1.91 (SPR) |
| 17-69-07-N451 | (B-Ala)-Sar10-A(Dap(Me))(D-Ala)NE(1Nal)(D-Ala) | Ki (h) = 3.7 nM |
| | CEDFYD(tBuGly)(Dap(Me)) | |
| 17-69-07-N461 | Ac(Dap(Me))(D-Ala)NE(1Nal)(D-Ala)CEDFYD | Ki (h) = 6.0 nM |
| | (tBuGly) (Dap(Me)) | |
| 17-69-07-N479 | Ac-A(Dap(Me))(D-Ala)NE(1Nal)(D-Ala)CEDFYD | Ki(h) = 15.6 nM |
| | (tBuGly)(Dap(Me)) | |
| 17-69-07-N474 | Ac-A(Dap(Me))(D-Ala)NE(1Nal)(D- | Ki (h) = 7.5 nM |
| | Ala)(Dap(Me))EDFYD (tBuGly)(Dap(Me)) | |

It can be seen that high affinity to MT1-MMP is achieved with these alkylamino-linked bicycle compounds according to the invention. Further studies showed full cross-reactivity of bicycle peptides according to the invention with dog, mouse/rat and human MT1-MMP. Further studies showed high specificity of bicycle peptides according to the invention, with no significant cross-reactivity with MMP1 ectodomain, MMP2 ectodomain, MMP15 ectodomain (hemopexin domain) or MMP16 hemopexin domain. The pharmacokinetics of bicycle peptides according to the invention were also determined to be similar to the corresponding bicycle peptides having three thioether linkages to the scaffold, but with slightly longer half-life in serum measurements for the bicycle peptides of the invention.

### Example 8

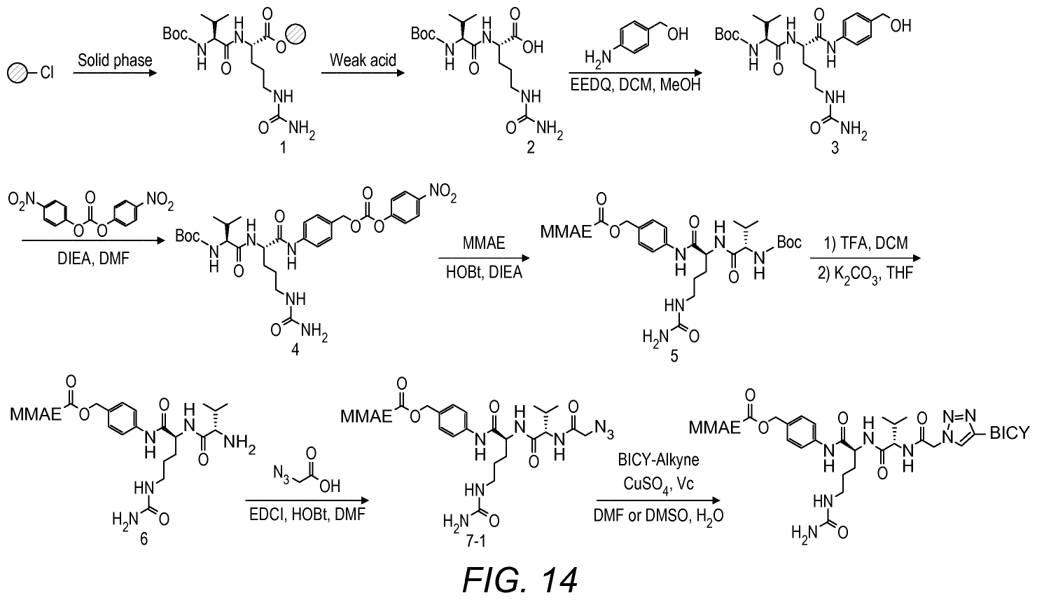

Bicycle peptide-drug conjugates (BCDs) in which bicycle peptides according to the invention are coupled to monomethyl auristatin E (MMAE) by a triazole cyclization reaction were prepared in accordance with the reaction scheme shown in FIG. 14. In addition to the triazole group, the linker groups in the conjugates include valine-citrulline (a cathepsin-cleavable group) and para-amino benzyl carbamate (PABC), a spacer group. The steps of the reaction scheme were performed as follows.

### General Procedure for Preparation of Compound 3

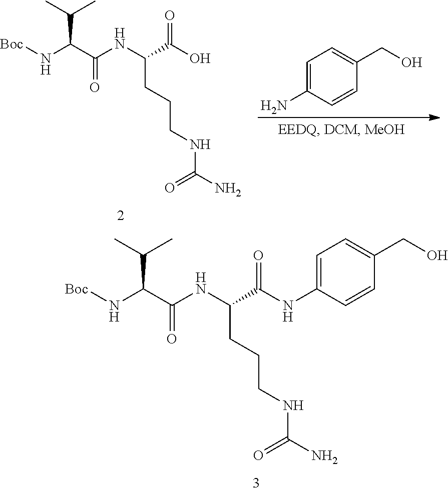 [SMILES: CC(C)[C@H](NC(=O)OC(C)(C)C)C(=O)N[C@@H](CCCNC(N)=O)C(=O)O ; Nc1ccc(CO)cc1 ; CC(C)[C@H](NC(=O)OC(C)(C)C)C(=O)N[C@@H](CCCNC(N)=O)C(=O)Nc1ccc(CO)cc1]

To a solution of compound 2 (30 g, 80 mmol) in DCM (300 mL) and MeOH (150 mL) was added 4-aminophenyl methanol (11 g, 88 mmol) and EEDQ (40 g, 160 mmol) in the dark. The mixture was stirred at 30° C. for 16 hr. TLC (DCM:MeOH=10/1, R_{f}=0.43) indicated compound 2 was consumed completely and many new spots formed. The reaction was clean according to TLC. The resulting reaction mixture was concentrated to give a residue, which was purified by flash silica gel chromatography (ISCO®; 330 g×3 SepaFlash® Silica Flash Column, Eluent of 0˜20% MeOH/Dichloromethane @ 100 mL/min). Compound 3 (20 g, 52% yield) was obtained as a white solid.

### General Procedure for Preparation of Compound 4

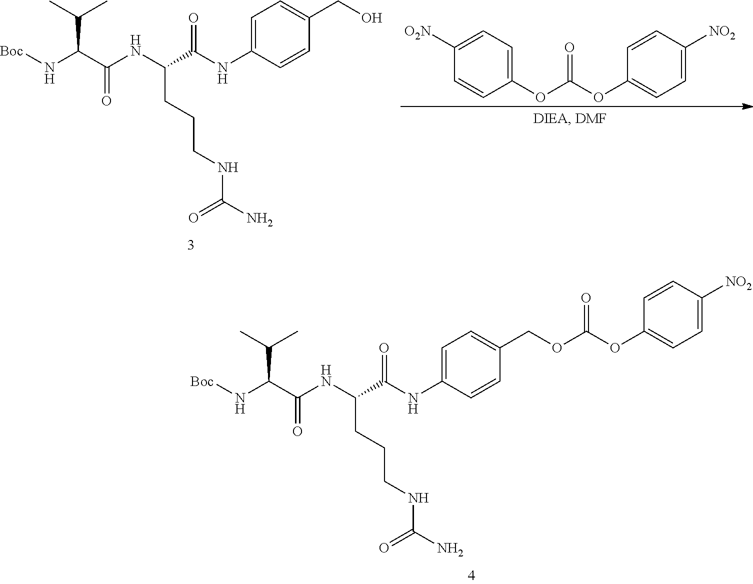 [SMILES: CC(C)[C@H](NC(=O)OC(C)(C)C)C(=O)N[C@@H](CCCNC(N)=O)C(=O)Nc1ccc(CO)cc1 ; O=C(Oc1ccc([N+](=O)[O-])cc1)Oc1ccc([N+](=O)[O-])cc1 ; CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1]

To a solution of compound 3 (5.0 g, 10.4 mmol) in DMF (40 mL) was added DIEA (5.4 g, 7.26 mL, 41.7 mmol) and bis(4-nitrophenyl) carbonate (12.7 g, 41.7 mmol). The mixture was stirred at 0° C. and under nitrogen for 1 hr. TLC (DCM:MeOH=10/1, R_{f}=0.66) indicated compound 3 was consumed completely and one new spot formed. The reaction was clean according to TLC and LCMS (ES8241-10-P1A, product: RT=1.15 min) showed the desired product was formed. The resulting reaction mixture was purified directly by prep-HPLC under neutral condition. Compound 4 (12 g, 60% yield) was obtained as a white solid.

### General Procedure for Preparation of Compound 5

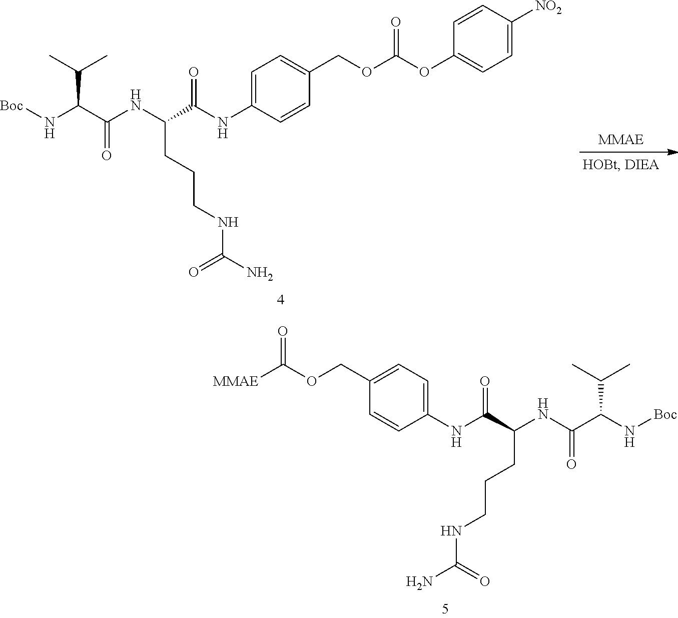

One batch of reaction was carried out as following: a solution of compound 4 (1.2 g, 1.68 mmol) in DMF (10 mL) was added DIEA (1.22 mL, 6.98 mmol,) under nitrogen atmosphere, the solution was stirred at 0° C. for 10 min, then HOBt (226 mg, 1.68 mmol) and MMAE (1.00 g, 1.40 mmol) were added thereto, the mixture was degassed and purged with N2 for 3 times, which was stirred at 35° C. for 16 hr. LC-MS (ES8396-1-P1A1, product: RT=1.19 min) showed compound 4 was consumed completely and one main peak with desired mass was detected. The resulting reaction mixture of five batches was combined in 1 L of beaker and 500 mL water was added, then a precipitate was formed and filtered to collect. The precipitate was triturated with EtOAc overnight. Compound 5 (5 g, 59% yield) was obtained as a white solid.

### General Procedure for Preparation of Compound 6

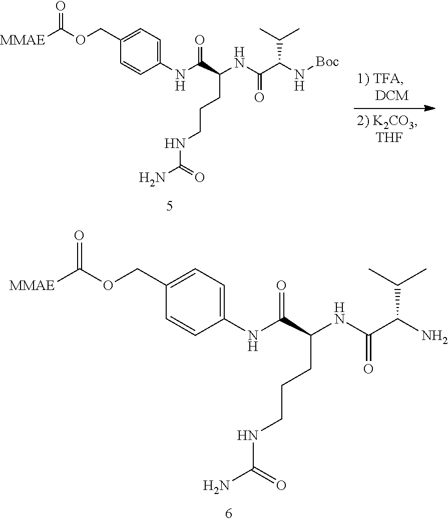

Compound 5 (3.3 g, 2.7 mmol) was dissolved in DCM (18 mL) in the presence of TFA (44 mmol, 3.5 mL), then the solution was stirred at 25° C. for 3 hr. Subsequently the reaction mixture was concentrated under reduced pressure to remove DCM and TFA to give a residue. The residue was dissolved in THF (20 mL), treated with K₂CO₃ (1.8 g, 13 mmol) and the mixture was further stirred at 25° C. for additional 12 hr. LC-MS (ES8396-2-P1B1, product: RT=1.04 min) showed one main peak with desired mass was detected. The reaction mixture was filtered and concentrated under reduced pressure to give a residue. The residue was dissolved in 10 mL of DMF and purified by prep-HPLC (neutral condition). Compound 6 (1.6 g, 53% yield) was obtained as a white solid.

### General Procedure for Preparation of Compound 7-1

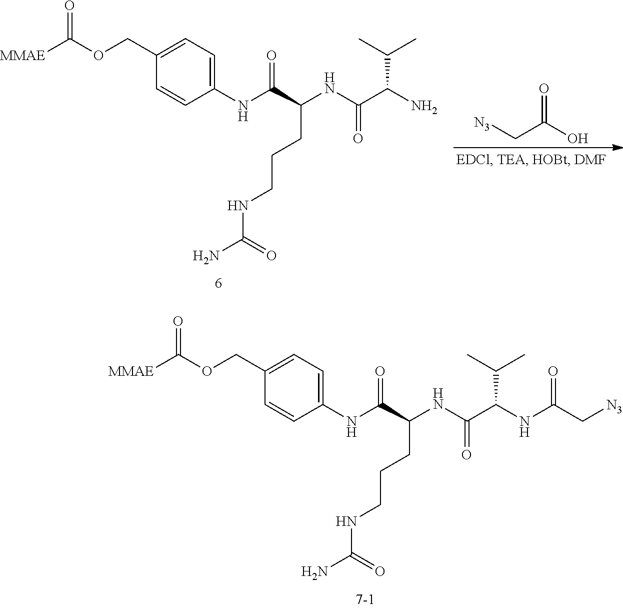

Compound 6 (1.2 g, 1.1 mmol) and 2-azidoacetic acid (162 mg, 1.6 mmol) were dissolved in DMF (10 mL). TEA (450 uL, 3.2 mmol), HOBt (217 mg, 1.6 mmol) and EDCI (307 mg, 1.6 mmol) were added to the solution under nitrogen, and the mixture was stirred at 0° C. for 30 min, then the mixture was warmed to 25° C. slowly with further stirring for 15.5 hr. LC-MS (ES8396-3-P1A, product: RT=1.04 min) showed compound 6 was consumed completely and one main peak with desired mass was detected. 2 mL of water was added to the reaction mixture to form a clear solution. Then the solution was purified directly by prep-HPLC under neutral condition. Compound 7-1 (0.9 g, 70% yield) was obtained as a white solid.

### General Procedure for Preparation of BT17BDC-53

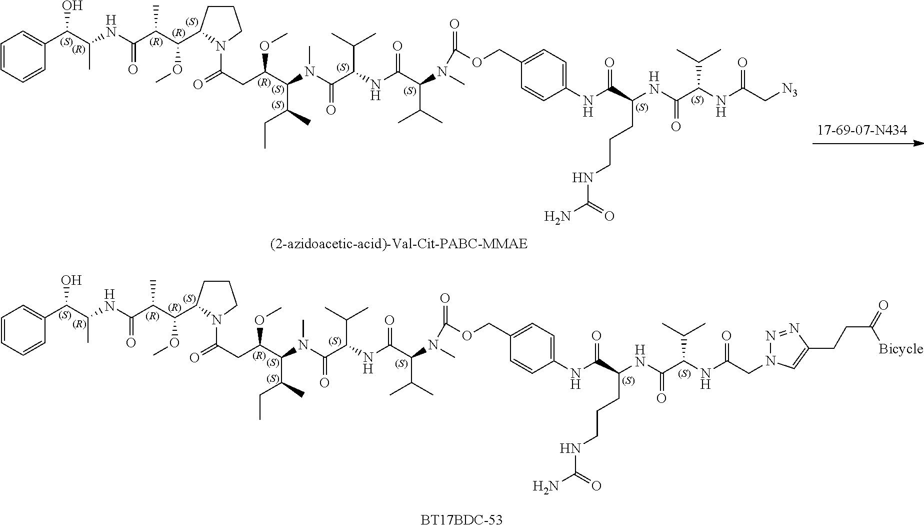

To a mixture of (2-azidoacetic-acid)-Val-Cit-PABC-MMAE (16 mg, 13.26 umol, 1 eq) and BICYCLE alkyne (17-69-07-N434, 30 mg, 11.09 umol, 0.8 eq) in DMF (3 mL) and H₂O (2 mL) was added CuI (1.26 mg, 6.63 umol, 0.5 eq). The mixture was stirred at 25° C. under N2 for 20 hr. LC-MS showed (2-azidoacetic-acid)-Val-Cit-PABC-MMAE was consumed completely and one main peak with desired MS was detected. The resulting reaction mixture was purified by prep-HPLC (TFA condition). Compound BT17BDC-53 (23.7 mg, 6.06 umol, 54.64% yield) was obtained as a white solid.

### General Procedure for Preparation of BT17BDC-59

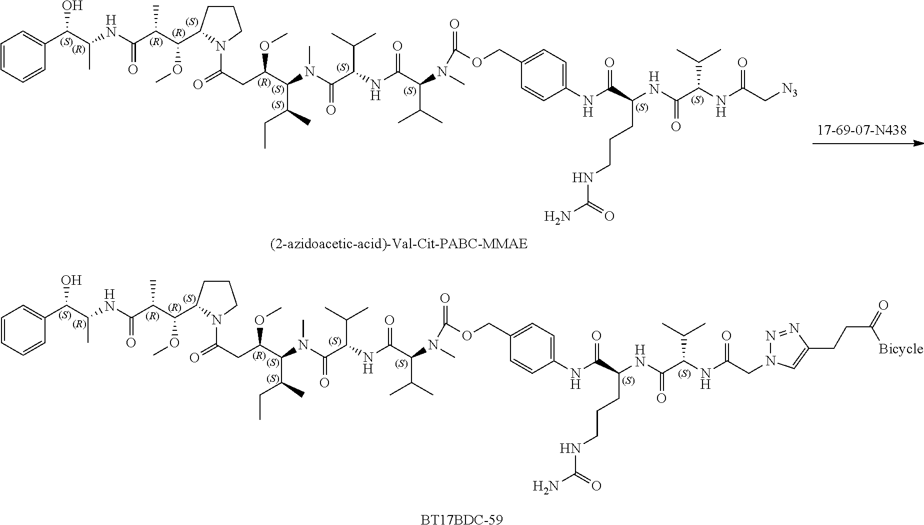

To a mixture of (2-azidoacetic-acid)-Val-Cit-PABC-MMAE (31 mg, 25.69 umol, 1.2 eq) and (17-69-07-N438, 40 mg, 20.8 umol, 1 eq) in DMF (3 mL) was added a solution of CuSO₄ (10.25 mg, 64.24 umol, 3 eq) in Water (0.4 mL) and a solution of Ascorbic Acid (37.71 mg, 214.12 umol, 10 eq) in Water (0.4 mL) under nitrogen. Then the mixture was stirred at 25° C. for 1 hr. LC-MS showed (2-azidoacetic-acid)-Val-Cit-PABC-MMAE was consumed completely and one main peak with desired MS was detected. The resulting reaction mixture was purified by prep-HPLC (TFA condition). BT17BDC-59 (26.7 mg, 8.53 umol, 41.02% yield) was obtained as a white solid.

### General Procedure for Preparation of BT17BDC61

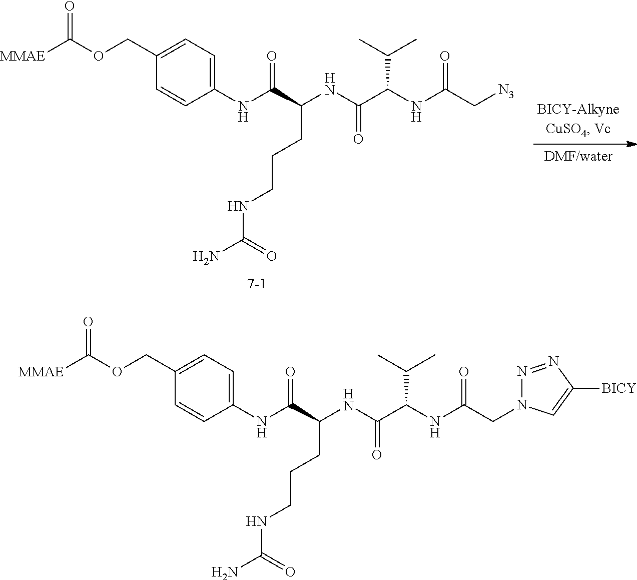

Compound 7-1 (250 mg, 207 umol) and BICY-ALKYNE 17-69-07-N450 (515 mg, 188 umol) were taken in an 50 mL of round flask, DMF (5 mL) was added, and followed by aqueous ascorbic acid solution (1 M, 1.88 mL) and aqueous CuSO₄ solution (1 M, 570 uL) under nitrogen atmosphere, then the mixture was stirred at 25° C. for 1 hr. LC-MS (ES8396-8-P1A, product: RT=1.03 min) showed BICY-ALKYNE was consumed completely and one main peak with desired mass was detected. The reaction mixture was filtered to remove the undissolved substance, filtrate was purified directly by prep-HPLC (TFA condition). BT17BDC61 (262 mg, 35% yield) was obtained as a white solid.

### General Procedure for Preparation of BT17BDC62

Compound 7-1 (250 mg, 207 umol) and BICY-ALKYNE 17-69-07-N443 (368 mg, 188 umol) were taken in a 50 mL of round flask. DMF (5 mL) was added, followed by adding an aqueous solution of ascorbic acid (1 M, 1.88 mL) and a aqueous solution of CuSO₄ (1 M, 570 uL). Then the mixture was stirred at 25° C. for 1 hr. LC-MS (ES8396-9-P1A, product: RT=1.07 min) showed BICY-ALKYNE was consumed completely and one main peak with desired mass was detected. The reaction mixture was filtered to remove the undissolved substance, The resulting filtrate was purified directly by prep-HPLC (TFA condition). BT17BDC62 (253 mg, 42% yield) was obtained as a white solid.

### Example 9

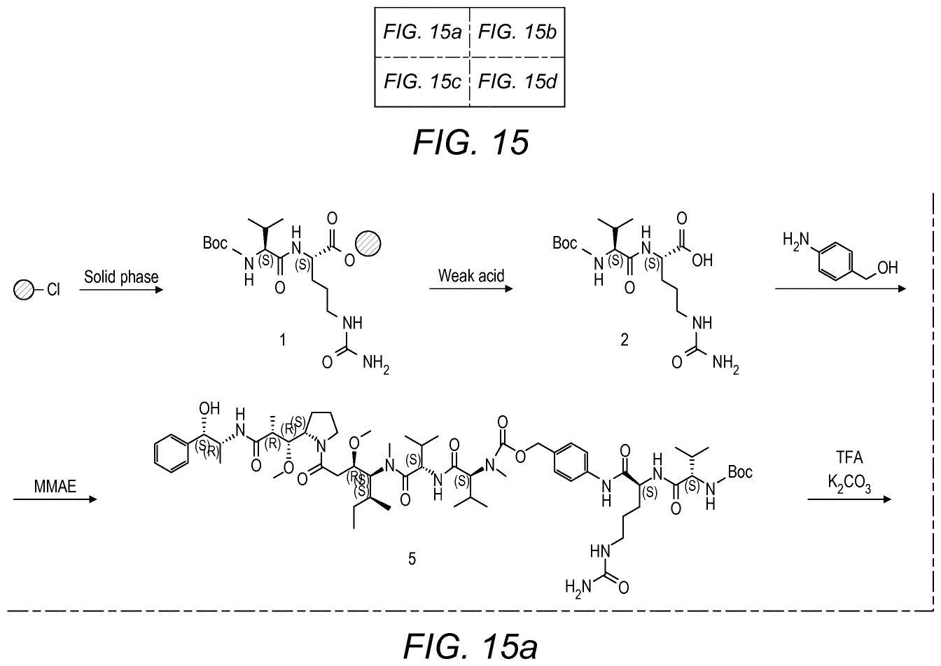

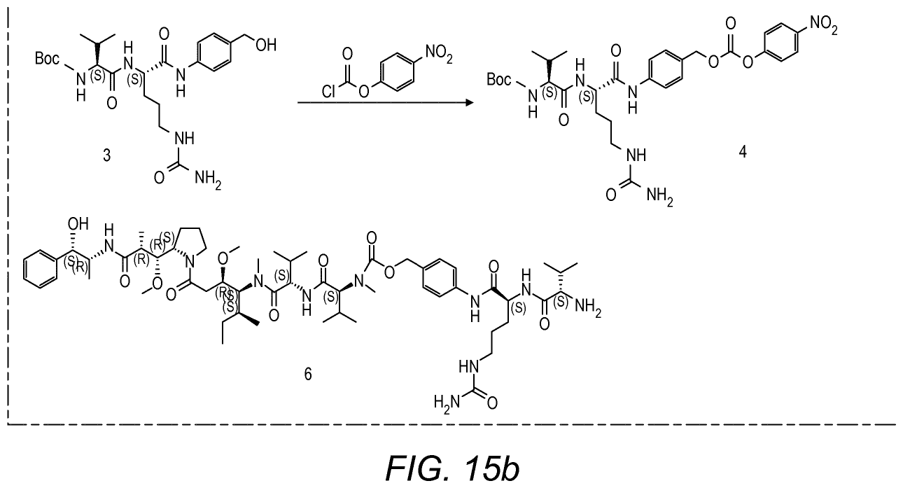

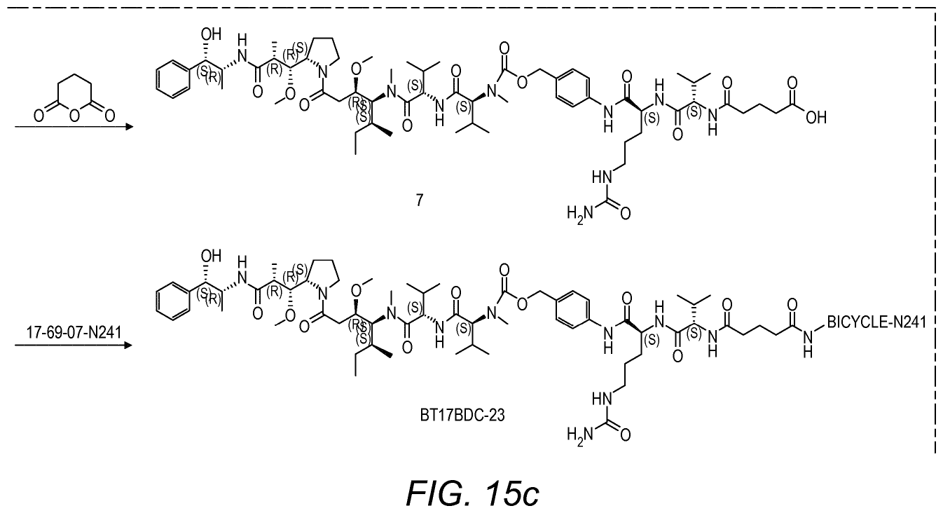

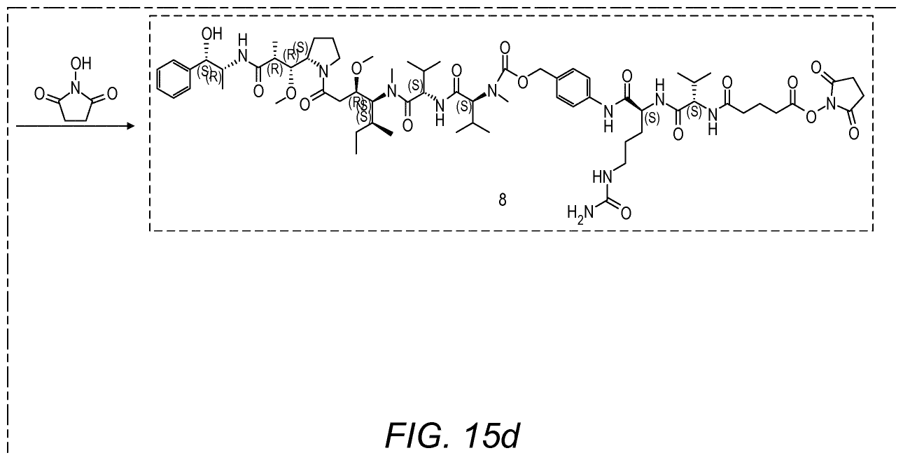

Bicycle-drug conjugates (BCDs) in which bicycle peptides according to the invention are coupled to monomethyl auristatin E (MMAE) by amide formation between a terminal glutaryl group of the linker and terminal amino of the peptide were prepared in accordance with the reaction scheme shown in FIG. 15. The steps of the reaction scheme were performed as follows.

### General Procedure for Preparation of Compound 3

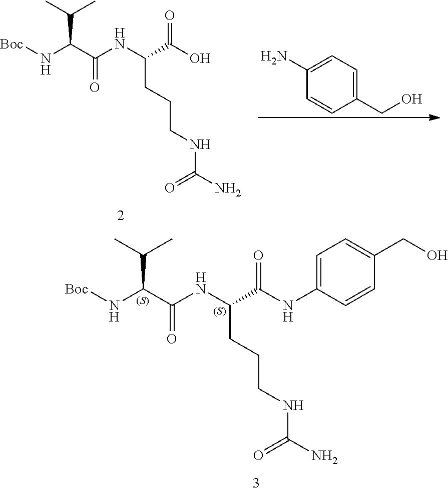

To a solution of Compound 2 (7.00 g, 18.70 mmol, 1.00 eq) in DCM (80.00 mL) and MeOH (40.00 mL) was added (4-aminophenyl)methanol (2.53 g, 20.56 mmol, 1.10 eq) and EEDQ (9.25 g, 37.39 mmol, 2.00 eq) in the dark. And the mixture was stirred at 25° C. for 8 hr. LC-MS showed Compound 2 was consumed completely and one main peak with desired MS was detected. The resulting reaction mixture was concentrated under reduced pressure to remove the solvent to give a residue. The residue was purified by flash silica gel chromatography (ISCO®; 120 g SepaFlash® Silica Flash Column, Eluent of 0-10% MeOH/DCM @ 85 mL/min). Compound 3 (7.00 g, 14.60 mmol, 78.06% yield) was obtained as a white solid.

### General Procedure for Preparation of Compound 4

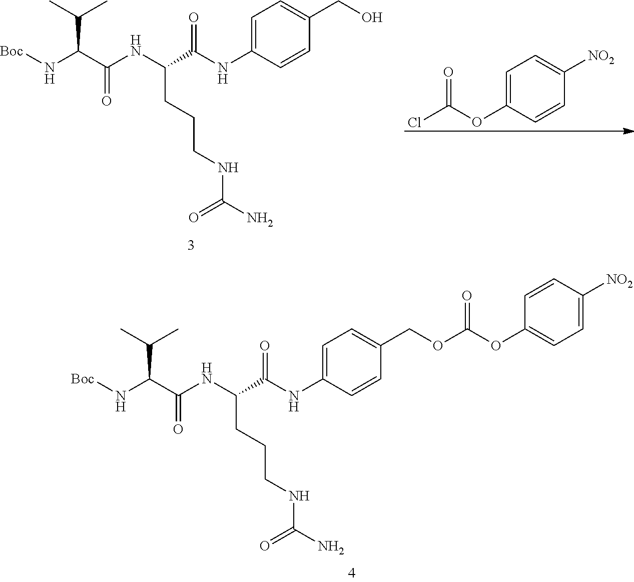 [SMILES: CC(C)[C@H](NC(=O)OC(C)(C)C)C(=O)N[C@@H](CCCNC(N)=O)C(=O)Nc1ccc(CO)cc1 ; O=C(Cl)Oc1ccc([N+](=O)[O-])cc1 ; CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1]

To a solution of Compound 3 (4.00 g, 8.34 mmol, 1.00 eq) and 4-nitrophenyl carbonochloridate (6.72 g, 33.36 mmol, 4.00 eq) in THF (20.00 mL) and DCM (10.00 mL) was added PYRIDINE (2.64 g, 33.36 mmol, 2.69 mL, 4.00 eq). And the reaction mixture was stirred at 25° C. for 5 hr. LC-MS showed Compound 3 was consumed completely and one main peak with desired MS was detected. The reaction mixture was concentrated under reduced pressure to give a residue, which was purified by flash silica gel chromatography (ISCO®; 120 g SepaFlash® Silica Flash Column, Eluent of 0-20% DM/MeOH@85 mL/min). Compound 4 (2.20 g, 3.41 mmol, 40.92% yield) was obtained as a white solid.

### General Procedure for Preparation of Compound 5

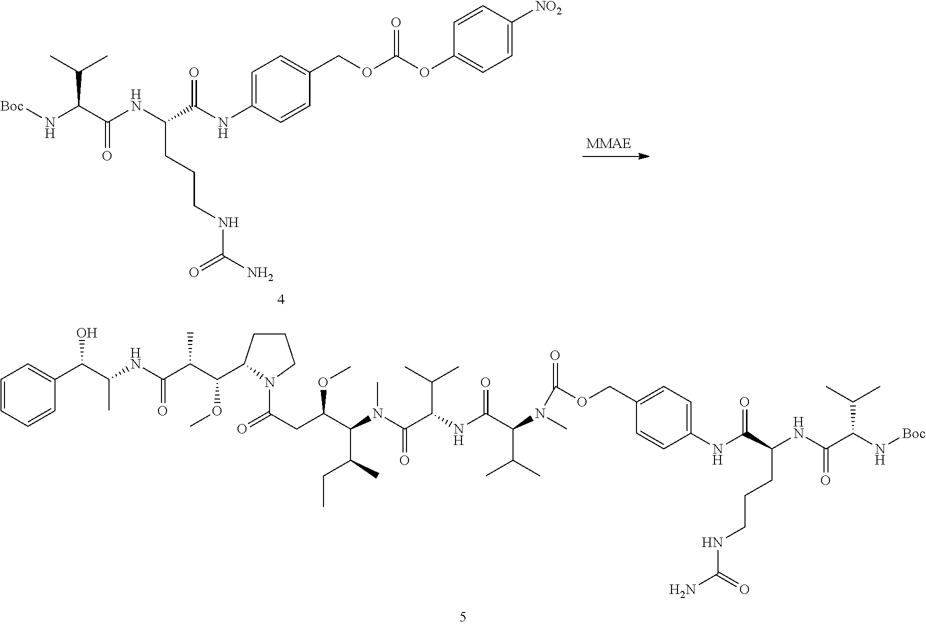 [SMILES: CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OC(C)(C)C)C(C)C)cc1)C(C)C]

A mixture of Compound 4 (500.00 mg, 775.59 umol, 1.00 eq) and DIEA (1.00 g, 7.76 mmol, 1.35 mL, 10.00 eq) in DMF (10.00 mL) was stirred under nitrogen at 0° C. for 30 min. And MMAE (445.49 mg, 620.47 umol, 0.80 eq) and HOBt (104.80 mg, 775.59 umol, 1.00 eq) was added to the above mixture. The reaction mixture was stirred under nitrogen at 0° C. for 10 min and at 30° C. for additional 18 hr. LC-MS showed Compound 4 was consumed completely and one main peak with desired MS was detected. The resulting reaction mixture was purified directly by flash C18 gel chromatography (ISCO®; 330 g SepaFlash® C18 Flash Column, Eluent of 0-50% MeCN/H₂O@85 mL/min). Compound 5 (400.00 mg, 326.92 umol, 42.15% yield) was obtained as a white solid.

### General Procedure for Preparation of Compound 6

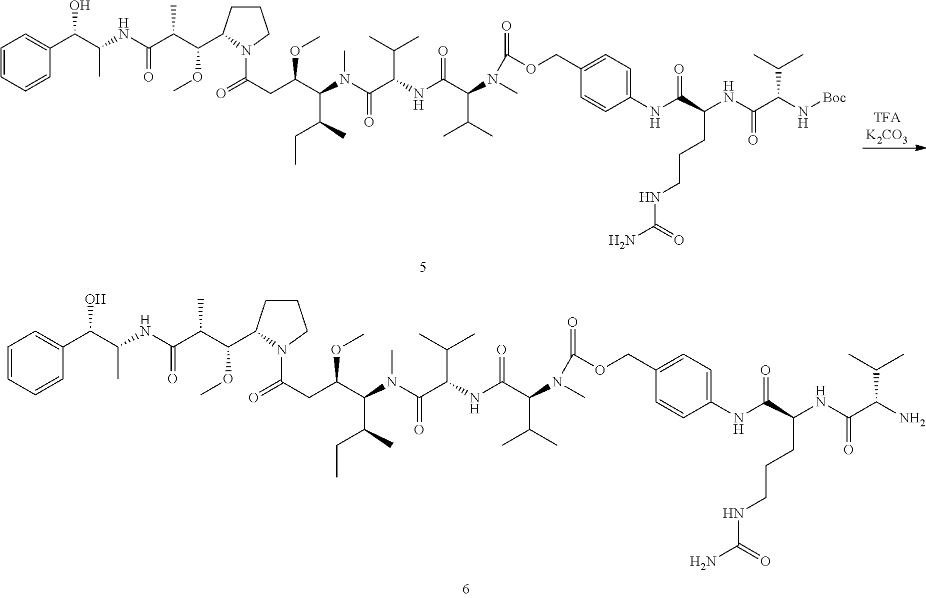 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OC(C)(C)C)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

To a solution of Compound 5 (430.00 mg, 351.44 umol, 1.00 eq) in DCM (36.00 mL) was added TFA (6.16 g, 54.03 mmol, 4.00 mL, 153.73 eq) and the mixture was stirred at 25° C. for 2 hr. The mixture was then concentrated under reduced pressure to give a residue, which was dissolved in THF (10.00 mL), and K₂CO₃ (1.21 g, 8.79 mmol, 25.00 eq) was added to the mixture. The reaction was stirred at 25° C. for 12 hr. LC-MS showed Compound 5 was consumed completely and one main peak with desired MS was detected. The resulting reaction mixture was filtered and the filtrate was concentrated under reduced pressure to give a residue, which was purified by flash C18 gel chromatography (ISCO®; 120 g SepaFlash® C18 Flash Column, Eluent of 0-50% MeCN/H₂O@85 mL/min). Compound 6 (290.00 mg, 258.14 umol, 73.45% yield) was obtained as a white solid.

### General Procedure for Preparation of Compound 7

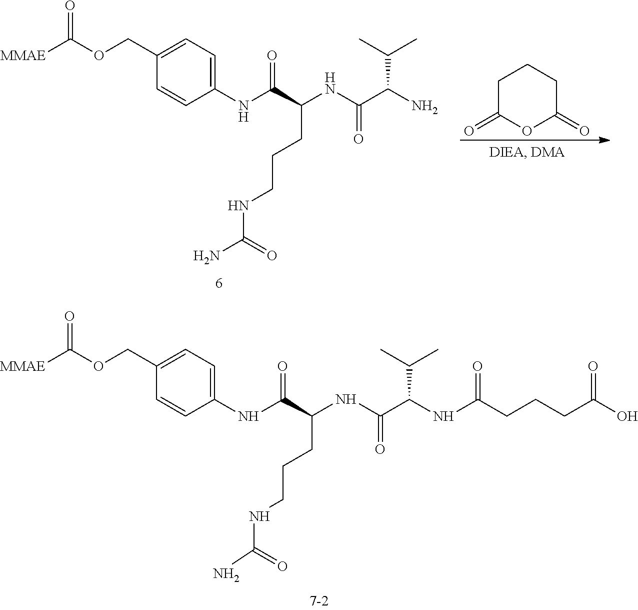

A vial containing (400 mg, 356 umol) was purged using a nitrogen balloon. Anhydrous DMA (5 mL) was added with stirring and the solution was cooled to 0° C. in an ice water bath. DIEA (130 uL, 712 umol) was then added and the reaction was stirred at 0° C. for 10 min. tetrahydropyran-2,6-dione (81 mg, 712 umol) was added and the ice bath was then removed. The reaction was stirred at 25° C. for 1 hr. LC-MS (ES8396-4-P1A, product: RT=1.08 min) showed compound 6 was consumed completely and one main peak with desired mass was detected. The mixture was diluted with 5 mL of water and then purified by prep-HPLC (neutral condition). Compound 7-2 (330 mg, 75% yield) was obtained as a white solid.

### General Procedure for Preparation of Compound 8

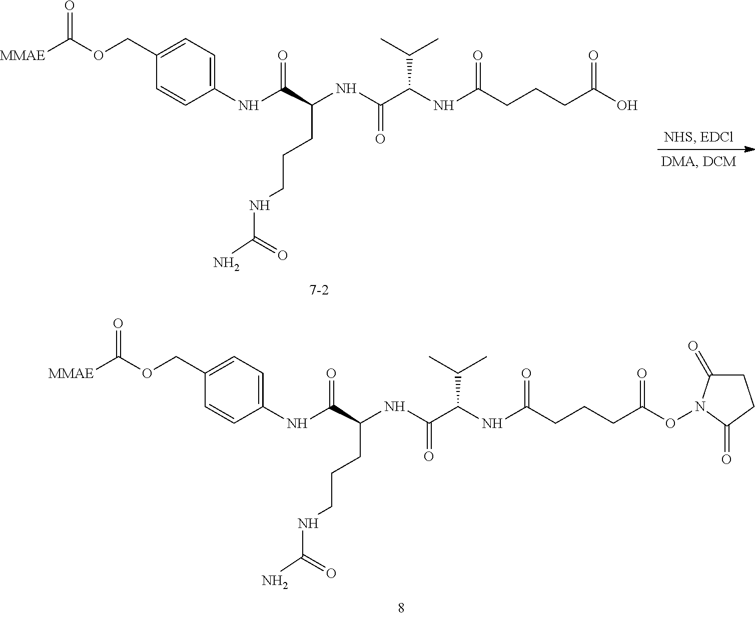

Compound 7-2 (330 mg, 267 umol) in anhydrous DMA (4.5 mL) and DCM (1.5 mL) was added HOSu (92 mg, 800 umol) under nitrogen with stirring for 10 min at 0° C. using an ice bath. Then EDCI (154 mg, 800 umol) was added to the mixture with further stirring at 25° C. for 16 hr. LC-MS (ES8396-5-P1A, product: RT=1.15 min) showed compound 7-2 was consumed completely and one main peak with desired mass was detected. The resulting reaction mixture was diluted with 5 mL of water and then purified by prep-HPLC (neutral condition). Compound 8 (250 mg, 70% yield) was obtained as a white solid.

### General Procedure for Preparation of BT17BDC68

A 50 mL of round bottom flask which contained BICY-NH₂ 17-69-07-N451 (80.0 mg, 30 umol) in DMA (4 mL) was purged using nitrogen balloon. DIEA (20 uL, 114 umol) was then added with stirring at 25° C. for 10 min. Compound 8 (40 mg, 30 umol) was then added and the reaction was stirred under a positive nitrogen atmosphere for 18 hr at 25° C. LC-MS (ES6635-127-P1A1, product: RT=1.06 min) showed compound 8 was consumed completely and one main peak with desired MS was detected. The resulting reaction mixture was purified by prep-HPLC (TFA condition). BT17BDC68 (33.9 mg, 29% yield) was obtained as a white solid.

### Example 10

The in vitro binding affinities of the bicycle peptide-drug conjugates prepared above were measured for MT1-MMP as previously described herein. The results were as follows.

| Compound | Parent | | Binding Affinity |
|---|---|---|---|
| ID | Bicycle ID | Linker | (Ki in nM) |
| BT17BDC-53 | 17-69-07-N434 | Triazole-2- | Human ki = 3.6, 2.5; |
| | | azidoacetyl- | rat/mouse ki = 1.8, 1.6 |
| | | ValCit-PABC | |
| BT17BDC-59 | 17-69-07-N438 | Triazole-2- | Human ki = 2.8, 2.7; |
| | | azidoacetyl- | rat/mouse ki = 1.8, 1.7 |
| | | ValCit-PABC | |
| BT17BDC-61 | 17-69-07-N450 | Triazole-2- | Human ki = 3.2, 2.9; |
| | | azidoacetyl- | rat/mouse ki = 1.8, 1.6 |
| | | ValCit-PABC | |
| BT17BDC-62 | 17-69-07-N443 | Triazole-2- | Human ki = 3.9, 3.3; |
| | | azidoacetyl- | rat/mouse ki = 2.1, 1.9 |
| | | ValCit- | |
| | | PABC-CO2- | |
| BT17BDC-68 | 17-69-07-N451 | glutaryl-Val-Cit | Human ki = 2.9, 3.3; |
| | | PABC | rat/mouse ki = 1.8, 1.6 |

It can be seen that in all cases the binding affinity of the bicycle peptides is maintained following conjugation to MMAE.

### Example 11

The plasma stability of BT17BDC-53 in mouse and human serums was studied. The conjugate was found to be stable (T_{1/2} greater than 50 hours at 4 μm concentration) in both mouse and human serums. The stability appears to be slightly greater than that of the corresponding conjugates in which the peptide is linked to the scaffold by three thioether linkages.

### Example 12

The in vivo efficacy against tumors of the bicycle peptide drug conjugates prepared above were evaluated as follows.

HT1080 tumor cells were maintained in vitro as a monolayer culture in EMEM medium supplemented with 10% heat inactivated fetal bovine serum at 37 C in an atmosphere of 5% CO₂ and air. The tumor cells were routinely subcultured twice weekly by trypsin-EDTA treatment. The cells growing in an exponential growth phase were harvested and counted for tumor inoculation.

BALB/c nude mice were inoculated subcutaneously at the right flank with HT1080 tumor cells (% x 10⁶) in 0.2 ml of PBS for tumor development. 39 animals were randomized when the average tumor volume reached 134 mm².

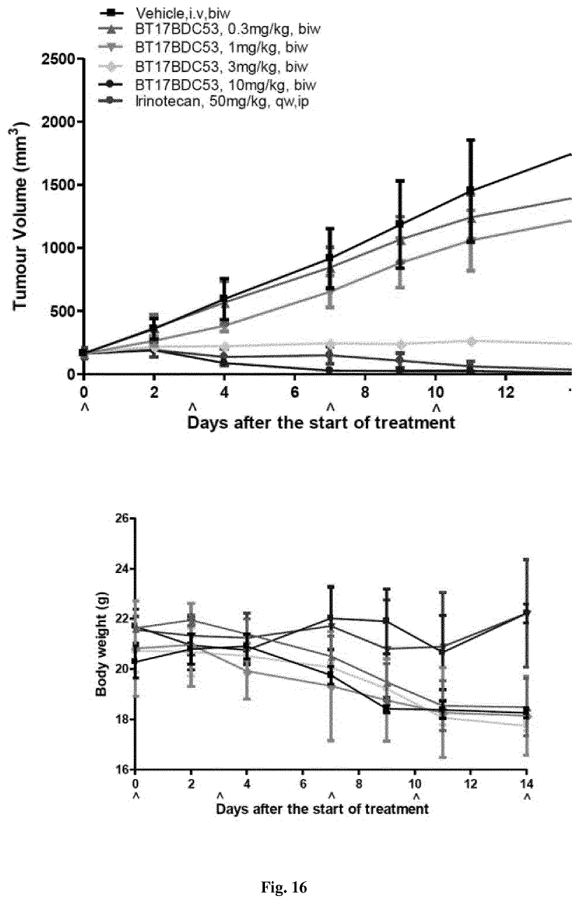

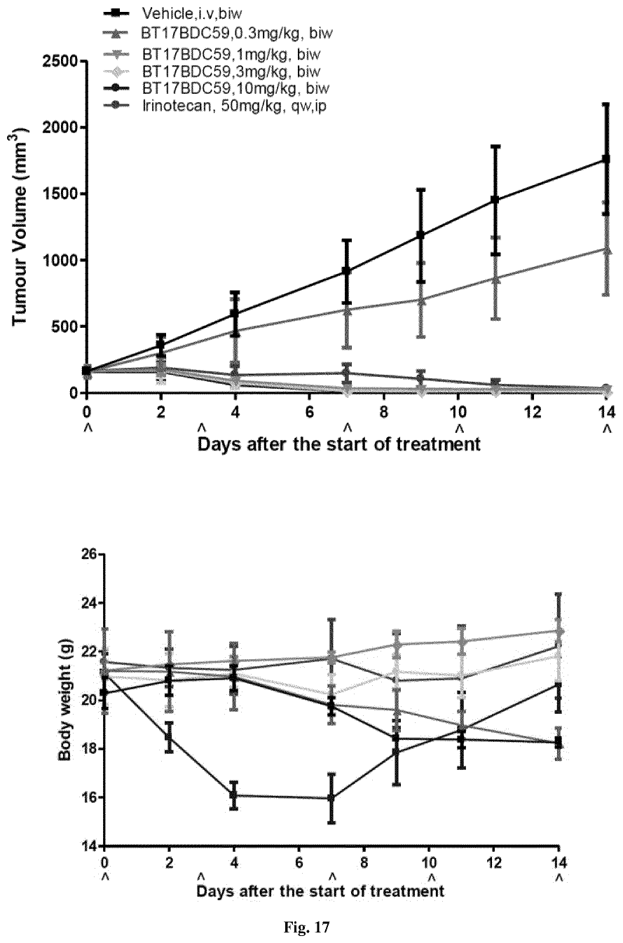

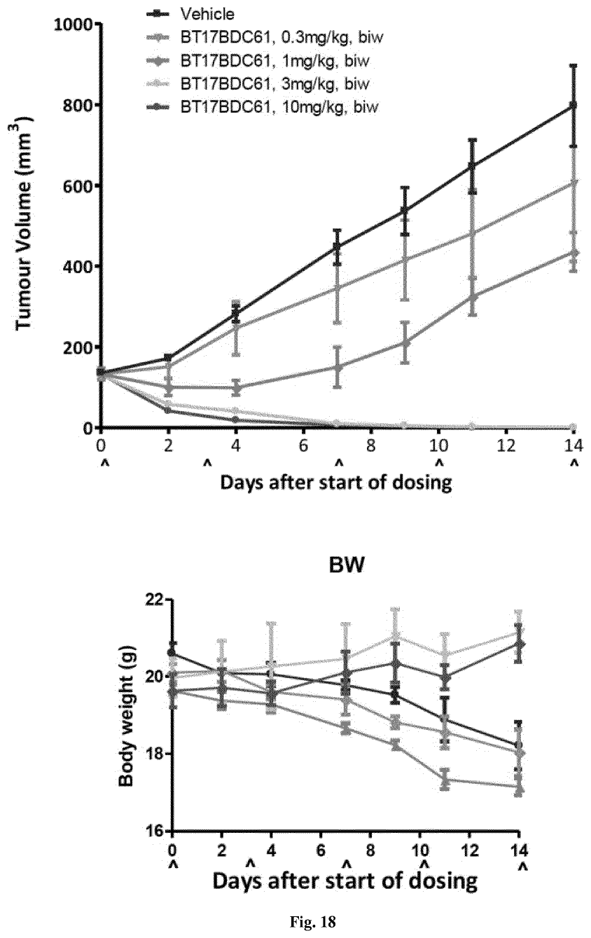

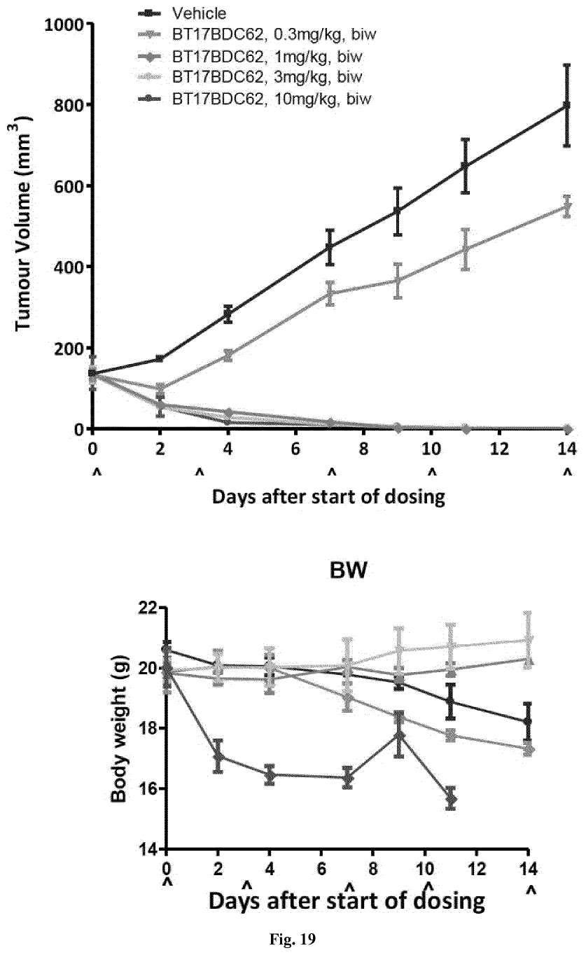

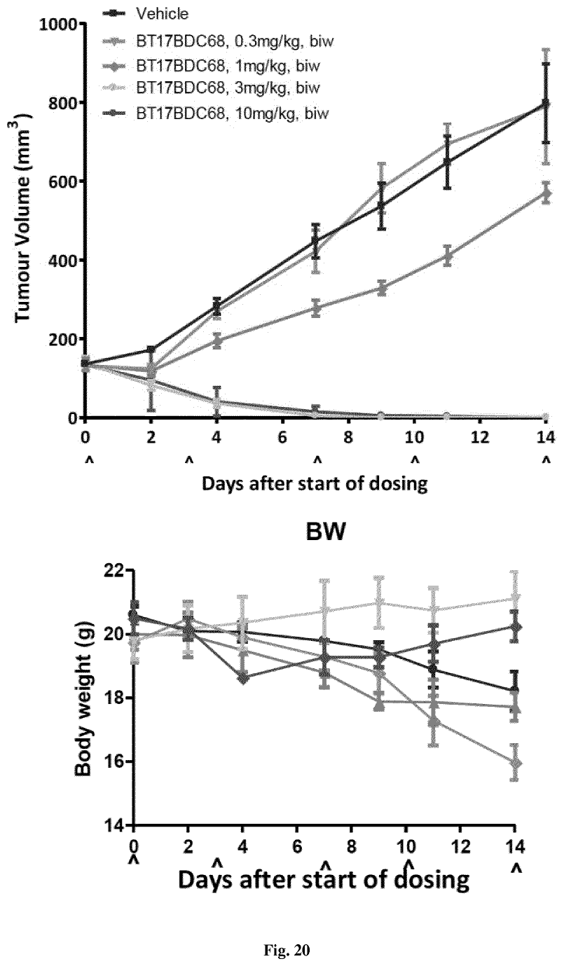

The BDC compounds were formulated at 0.03 mg/ml in a vehicle buffer containing 25 mM histidine and 10% Sucrose. The formulations were administered twice weekly (biw) at 0.3, 1, 3 and 10 mg/kg. Tumor volume and body weight were measured up to 14 days from the first dosing. The results are shown in FIGS. 16-20.

The results show that all five of the conjugates that were tested exhibit strong dose-dependent tumor inhibition. At doses of 3 mg/kg and 10 mg/kg the tumors appeared to be completely eradicated. BT17BDC53, 61 and 68 were well tolerated up to 10 mg/kg. BT17BDC62 was tolerated up to about 5 mg/kg. BT17BDC59 was tolerated up to 3 mg/kg. This suggests that the presence of the N-terminal Sar10 spacer in BT17BDC53, 61 and 68 reduces the systemic toxicity of the conjugates.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A peptide ligand specific for MT1-MMP comprising a polypeptide comprising three residues selected from cysteine, L-2,3-diaminopropionic acid (Dap), N-beta-alkyl-L-2,3-diaminopropionic acid (N-AlkDap) and N-beta-haloalkyl-L-2,3-diaminopropionic acid (N-HAlkDap), the said three residues being separated by at least two loop sequences, and a molecular scaffold, the peptide being linked to the scaffold by covalent alkylamino linkages with the Dap or N-AlkDap or N-HAlkDap residues of the polypeptide and by thioether linkages with the cysteine residues of the polypeptide when the said three residues include cysteine, such that two polypeptide loops are formed on the molecular scaffold, wherein the peptide ligand comprises an amino acid sequence of formula (II):
(II)
(SEQ ID NO: 1)
-A₁-X₁-U/O₂-X₃-X₄-G₅-A₂-E₆-D₇-F₈-Y₉-X₁₀-X₁₁-A₃-
or a pharmaceutically acceptable salt thereof;
wherein:
A₁, A₂, and A₃ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of A₁, A₂, and A₃ is Dap, N-AlkDap or N-HAlkDap;
X represents any amino acid residue;
U represents a polar, uncharged amino acid residue selected from N, C, Q, M, S and T; and
O represents a non-polar aliphatic amino acid residue selected from G, A, I, L, P and V.

2. The peptide ligand as defined in claim 1, wherein Xi is selected from any one of the following amino acids: Y, M, F or V, such as Y, M or F, in particular, Y or M, more particularly Y.

3. The peptide ligand as defined in claim 1, wherein U/O₂ is selected from a U, such as an N, or an O, such as a G.

4. The peptide ligand as defined in claim 1, wherein X₃ is selected from U or Z, wherein U represents a polar, uncharged amino acid residue selected from N, C, Q, M, S and T and Z represents a polar, negatively charged amino acid residue selected from D or E, in particular the U at position 3 is selected from Q or the Z at position 3 is selected from E.

5. The peptide ligand as defined in claim 1, wherein X₄ is selected from J, wherein J represents a non-polar aromatic amino acid residue selected from F, W and Y.

6. The peptide ligand as defined in claim 1, wherein X₁₀ is selected from Z, wherein Z represents a polar, negatively charged amino acid residue selected from D or E, such as D.

7. The peptide ligand as defined in claim 1, wherein X₁₁ is selected from O, wherein O represents a non-polar aliphatic amino acid residue selected from G, A, I, L, P and V, such as I.

8. The peptide ligand as defined in claim 1, wherein the Bicycle of formula (II) is a compound of formula (IIa):
(IIa)
(SEQ ID NO: 6)
-A₁-Y/M/F/V-U/O-U/Z-J-G-A₂-E-D-F-Y-Z-O-A₃-
wherein U, O, J and Z are as defined hereinbefore; or
a compound of formula (IIb):
(IIb)
(SEQ ID NO: 7)
-A₁-Y/M/F/V-N/G-E/Q-F-G-A₂-E-D-F-Y-D-I-A₃-;
or
a compound of formula (IIc):
(IIc)
(SEQ ID NO: 8)
-A₁-Y/M/F-N/G-E/Q-F-G-A₂-E-D-F-Y-D-I-A₃-;
or
a compound of formula (IId):
(IId)
(SEQ ID NO: 9)
-A₁-Y/M-N-E/Q-F-G-A₂-E-D-F-Y-D-I-A₃-;
or
a compound of formula (IIe):
(IIe)
(SEQ ID NO: 2)
-A₁-Y-N-E-F-G-A₂-E-D-F-Y-D-I-A₃-(17-69-07).

9. The peptide ligand as defined in claim 1, wherein the Bicycle of formula (II) comprises a sequence selected from:
(SEQ ID NO: 2)
-A₁-Y-N-E-F-G-A₂-E-D-F-Y-D-I-A₃-(17-69-07);
(SEQ ID NO: 10)
-A₁-M-N-Q-F-G-A₂-E-D-F-Y-D-I-A₃-(17-69-12);
(SEQ ID NO: 11)
-A₁-F-G-E-F-G-A₂-E-D-F-Y-D-I-A₃-(17-69-02);
(SEQ ID NO: 12)
-A₁-V-N-E-F-G-A₂-E-D-F-Y-D-I-A₃-(17-69-03);
(SEQ ID NO: 13)
-A₁-F-N-E-F-G-A₂-E-D-F-Y-D-I-A₃-(17-69-04);
(SEQ ID NO: 14)
-A₁-Y-N-E-Y-G-A₂-E-D-F-Y-D-I-A₃-(17-69-07-N057);
and
(SEQ ID NO: 15)
-A₁-Y-N-E-W-G-A₂-E-D-F-Y-D-I-A₃-(17-69-44-N002),
such as:
(SEQ ID NO: 2)
-A₁-Y-N-E-F-G-A₂-E-D-F-Y-D-I-A₃-(17-69-07);
and
(SEQ ID NO: 10)
-A₁-M-N-Q-F-G-A₂-E-D-F-Y-D-I-A₃-(17-69-12),
in particular:
(SEQ ID NO: 2)
-A₁-Y-N-E-F-G-A₂-E-D-F-Y-D-I-A₃-(17-69-07),
most particularly:
the Dap homologue designated as SEQ ID 16 ((bAla)-Sar10-AA₁(D-Ala)NE(1Nal)(D-Ala) A₂EDFYD(tBuGly)A₃;
and the Dap homologue designated as SEQ ID 17 AA₁(D-Ala)NE(1Nal)(D-Ala) A₂EDFYD(tBuGly)A₃.

10. The peptide ligand as defined in claim 1, wherein two of A₁, A₂ and A₃ are selected from Dap, N-AlkDap or N-HAlkDap, and the third one of A₁, A₂ and A₃ is cysteine, preferably wherein A₂ is cysteine.

11. The peptide ligand as defined in claim 1, wherein A₁, A₂ and A₃ are each N-AlkDap or N-HAlkDap,

12. The peptide ligand as defined in claim 1, wherein one or more tyrosine residues is replaced by a phenylalanine residue.

13. The peptide ligand as defined in claim 1, which additionally comprises one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more hydrophobic amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the α-carbon of one or more amino acid residues with another chemical group, and post-synthetic bioorthogonal modification of amino acids such as cysteine, lysine, glutamate and tyrosine with suitable amine, thiol, carboxylic acid and phenol-reactive reagents.

14. (canceled)

15. The peptide ligand as defined in claim 13, wherein the N-terminal modification comprises the addition of a molecular spacer group which facilitates the conjugation of effector groups and retention of potency of the bicyclic peptide to its target, such as an Ala, G-Sar10-A group or bAla-Sar10-A group.

16. The peptide ligand as defined in claim 13, wherein said N-terminal and/or C-terminal modification comprises addition of a cytotoxic agent.

17-27. (canceled)

28. A linear peptide comprising an amino acid sequence of formula (II) as defined in claim 1.

29. (canceled)

30. A drug conjugate comprising a peptide ligand as defined in claim 1, conjugated to one or more effector and/or functional groups.

31. The drug conjugate as defined in claim 30, wherein the effector and/or functional groups comprises a cytotoxic agent or a metal chelator.

32. The drug conjugate as defined in claim 31, wherein the cytotoxic agent is linked to the bicyclic peptide by a cleavable bond, such as a disulphide bond.

33. The drug conjugate as defined in claim 31, wherein the cytotoxic agent is selected from DM1 or MMAE.

34. The drug conjugate as defined in claim 31, wherein the drug conjugate has the following structure: 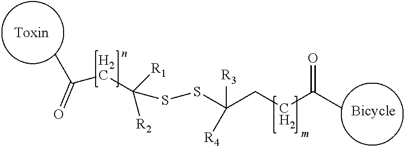
wherein R₁, R₂, R₃ and R₄ represent hydrogen or C1-C6 alkyl groups;
Toxin refers to any suitable cytotoxic agent;
n represents an integer selected from 1 to 10; and
m represents an integer selected from 0 to 10.

35. The drug conjugate as defined in claim 34, wherein either: R₁, R₂, R₃ and R₄ are all H; or R₁, R₂, R₃ are all H and R₄=methyl; or R₁, R₂=methyl and R₃, R₄=H; or R₁, R₃=methyl and R₂, R₄=H; or R₁, R₂=H and R₃, R₄=C1-C6 alkyl.

36. The drug conjugate as defined in claim 31, wherein the drug conjugate has the following structure: 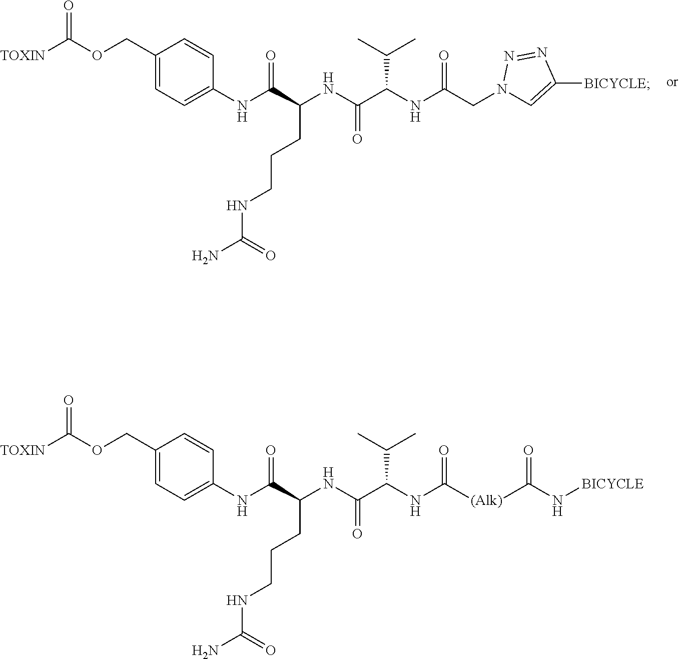
wherein (alk) is a linear or branched alkylene group of formula CₙH₂ₙ wherein n is from 1 to 10.

37. (canceled)
